(19) United States
(12) Patent Application Publication
(10) Pub. No.: US 2025/0382667 A1
(43) Pub. Date: Dec. 18, 2025

(54) IDENTIFYING PATIENT CONDITIONS BY TRANSFORMING NUCLEIC ACID SEQUENCE DATA INTO ALTERNATE DOMAINS

(71) Applicant: Foundation Medicine, Inc., Cambridge, MA (US)

(72) Inventors: Zoe R. Fleischmann, Jamaica Plain, MA (US); Ethan S. Sokol, Somerville, MA (US); Cai John, Somerville, MA (US)

(73) Assignee: Foundation Medicine, Inc., Cambridge, MA (US)

(21) Appl. No.: 19/237,646

(22) Filed: Jun. 13, 2025

Related U.S. Application Data
(60) Provisional application No. 63/659,764, filed on Jun. 13, 2024.

(51) Int. Cl. C12Q 1/6869; C12Q 1/6886; G06N 3/0464; G16B 30/00; G16H 50/20

(52) U.S. Cl. CPC C12Q 1/6869; C12Q 1/6886; G06N 3/0464; G16B 30/00; G16H 50/20

(57) ABSTRACT

Techniques for identifying conditions of a subject are described. An example method includes identifying sequence read data of a sample obtained from a subject. The sequence read data is in a spatial domain corresponding to genomic position. The example method further includes generating transformed data by transforming the sequence read data into an alternative domain; generating input features based on the transformed data; and classifying, using a classifier, a condition of the subject based on the input features.

### CROSS-REFERENCE TO RELATED APPLICATION

This application claims priority to U.S. Provisional Application No. 63/659,764, which was filed on Jun. 13, 2024 and is incorporated by reference herein in its entirety.

### BACKGROUND

Many individuals rely on genetic testing to identify whether they have, or are predicted to develop, various health related conditions. In some cases, single gene testing can be used to assess whether an individual has a particular genetic mutation that is relevant to whether the individual has a genetic disorder or a propensity for disease. Multiple genes, in some cases, can be tested in order to provide even greater context into the individual's health. Whole exome sequencing (WES) and whole genome sequencing (WGS) can provide even further context.

Extensive genomic sequencing methodologies, such as those utilizing sequence read data obtained by WGS, can result in a substantial amount of data for analysis. It may be difficult to process this substantial amount of data, directly, to accurately identify whether an individual has a particular condition, such as a type of cancer. For instance, a substantial amount of processing resources may be utilized in order to identify a condition of a subject using sequence read data. Moreover, some conditions are not apparent by evaluating sequence read data directly.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for predicting a condition of a subject by transforming genomic information of the subject.

FIG. 2 illustrates example signaling for selecting features for classifying a condition of a subject based on transformed genomic information of the subject.

FIG. 3 illustrates an example environment for training and utilizing a predictive model to identify a condition of a subject.

FIG. 4 illustrates an example of training data utilized to train one or more machine learning (ML) models.

FIG. 5 illustrates an example report summarizing predicted conditions of a subject.

FIG. 6 illustrates an example environment for sequencing various nucleic acid molecules.

FIG. 7 illustrates an example environment illustrating circulating tumor DNA (ctDNA), which can be utilized to a condition of a subject.

FIG. 8 illustrates an example process for identifying a condition of a subject using transformed data.

FIG. 9 illustrates one or more devices configured to perform various operations described herein.

FIG. 10 illustrates an example of endpoint data for nucleic acid samples in first liquid biopsy samples (upper) and endpoint data for nucleic acid samples in second liquid biopsy samples (lower), wherein the first liquid biopsy samples have relatively high percentages of prostate cancer tumor DNA and the second liquid biopsy samples have relatively low percentages of prostate cancer tumor DNA.

FIG. 11 illustrates shifts in periodicity between the liquid biopsy samples of FIG. 10.

FIG. 12 compares raw signal to cross-correlations of fragmentomics data (e.g., endpoint counts) for high shed prostate cancer samples, other non-prostate cancer high shed samples, and samples without circulating tumor fragments.

FIG. 13 illustrates the nearly 400 regions of the X chromosome that were analyzed in the Example for features that are predictive of prostate cancer status.

FIG. 14 illustrates results of logistic regression, showing these features result in reasonable predictive performance for assessing prostate cancer status.

Some of the drawings submitted herewith may be better understood in color. Applicant considers the color versions of the drawings as part of the original submission and reserves the right to present color images of the drawings in later proceedings.

### DETAILED DESCRIPTION

Various implementations of the present disclosure relate to techniques for predicting health-related conditions based on transformed nucleic acid sequencing data. In various cases, nucleic acid molecules are obtained from a subject. In some cases, the nucleic acid molecules include DNA fragments obtained from a liquid biopsy sample. Sequence read data is generated by sequencing the nucleic acid molecules. In various cases, the sequence read data includes at least one dimension that represents a position of the sequenced nucleic acid molecules in a reference genome (also referred to as a “genomic position”), such that the sequence read data is in a spatial domain.

In various implementations of the present disclosure, the sequence read data is transformed into an alternate domain. For instance, the sequence read data may be transformed into a frequency or wavelet domain by performing an appropriate transform on the sequence read data. The transformed sequence read data (also referred to as “transformed data”) exhibits various features of the subject that are difficult to impossible to ascertain in the original domain of the sequence read data. These features, for instance, are predictive of the health-related conditions. According to various examples, the features of the transformed data are used to determine a condition of the subject. For instance, the features may be input into a predictive model that is configured to determine whether the subject has the condition. In various cases, indications of the condition of the subject are reported to the subject directly or to a care provider that is responsible for the subject.

Various types of health-related conditions can be predicted using various techniques described herein. In some cases, these techniques are used to determine whether the subject has a disease. In particular examples, these techniques are used to determine whether the subject has cancer, or whether the subject has a particular type of cancer. Conditions related to pathogenic conditions can also be determined, such as a predicted effective therapy to treat the pathogenic condition, a predicted stage of the pathogenic condition, or a predicted grade of the pathogenic condition. Non-pathogenic conditions can also be predicted using implementations of the present disclosure. For instance, the transformed data can be used to predict a general health of the subject, a risk of developing a disease (e.g., a type of cancer), a genomic age of the subject, a predicted survivability of the subject.

Implementations of the present disclosure provide significant improvements to the technical field of medical diagnostics and treatment. Utilizing transformed sequence read data may greatly enhance the accuracy of predictions of health-related conditions based solely on nucleic acid analyses. In some cases, the techniques described herein can be used to predict whether a subject has a particular condition with high (e.g., 90%, 95%, 99%, or the like) accuracy using nucleic acid molecules that are obtained using a minimally invasive liquid biopsy process. Accordingly, the subject and care providers may make informed decisions about the subject's health without the subject being subjected to highly invasive procedures, such as surgeries (e.g., tissue biopsy procedures). In some examples, the transformed sequence read data may identify new conditions that are not otherwise apparent using previous biomarkers or genomic analyses.

Various analyses described herein cannot be performed in the human mind, or by pen and paper. For example, it would not be possible to transform sequence read data representing numerous (e.g., hundreds, thousands, etc.) of bases in a sample into an alternate domain (e.g., a frequency domain) solely in the mind of a human.

### Example Definitions

As used herein, the terms “deoxyribonucleic acid,” “DNA,” “DNA molecule.” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end.” “3-prime end.” and their equivalents, may refer to a terminus of a single-stranded nucleotide polymer that includes a base whose third carbon in its deoxyribose or ribose is bound to a hydroxyl group while being unbound to another base.

As used herein, the terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene.” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome.” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome.” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the terms “end motif,” “terminal sequences,” and their equivalents, may refer to a sequence of nucleotides extending from a 3′ or 5′ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

As used herein, the term “promoter,” and its equivalents, may refer to a portion of a DNA molecule that binds one or more proteins in order to initiate transcription of a gene. For example, the promotor is located “upstream” of the gene. For example, the promotor is located between 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs.

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “condition,” and its equivalents, may refer to the state of an individual's health. A condition may refer to a positive state (e.g., a visual acuity that is better than 20/20 vision, nonpathological hypotension, etc.), a normal state (e.g., a normal blood pressure), a negative state (e.g., a pathological condition, such as cancer), or any combination thereof.

As used herein, the term “pathological condition,” “pathology,” “disease,” and their equivalents, may refer to an abnormal anatomical, physiological, or psychological condition that reduces one or more functional abilities below a typical efficiency. As a result of a pathological condition, a subject may have an impaired function, pain, reduced life expectancy, or some other negative health consequence.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located. Cancer is a type of pathological condition.

As used herein, the terms “tumor.” “neoplasm.” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject's body.

As used herein, the terms “liquid biopsy.” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy.” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject.” and its equivalents, may refer to a human or non-human animal. A subject that is receiving care from at least one care provider may be referred to as a “patient.”

As used herein, the term “variant,” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion.” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation.” “CNA.” “copy number variation,” “CNV,” and their equivalents, can refer to a portion of a reference sequence that is repeated.

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing.” and its equivalents, may refer to a process of identifying the order and identity of monomers in a polymer chain, such as the order and identity of nucleotides in a DNA or RNA molecule. The terms “whole genome sequencing.” “WGS,” “full genome sequencing” and their equivalents, may refer to the process of sequencing an entire genome of a subject, including the introns and exons of the genes of the subject. The terms “whole exome sequencing.” “WES,” and their equivalents, may refer to the process of sequencing all exomes of a subject. The term “targeted sequencing.” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next generation sequencing (NGS). An apparatus configured to perform NGS is referred to as a “next generation sequencer.” In various cases, sequencing is performed on physical molecules (e.g., RNA or DNA) and is used to generate data.

As used herein, the terms “massive parallel sequencing.” “massively parallel sequencing.” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing.” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space. The electrical signal, for instance, can be detected by sensors disposed in the first space and the second space.

As used herein, the term “locus,” and its equivalents, may refer to a specific location of one or more nucleic acid molecules on a chromosome, genome, pangenome, or the like. In some cases, a locus refers to a location of a gene, genetic marker, or other sequence is located on a chromosome. The plural form of “locus” is “loci.”

As used herein, the term “endpoint,” and its equivalents, may refer to one or more bases located at a terminus of a nucleic acid molecule fragment. When a fragment is aligned with a reference genome, a “right” or “lower” endpoint of the fragment may correspond to the largest coordinate in the reference genome that is aligned with the fragment. A “left” or “upper” endpoint of the fragment may correspond to the smallest coordinate in the reference genome that is aligned with the fragment.

As used herein, the term “genomic position,” and its equivalents, may refer to a molecular location of one or more base pairs within a reference genome. In some cases, the molecular location is defined by the chromosome on which the base pair(s) is located, the arm of the chromosome on which the base pair(s) is located, the distance (e.g., in base pairs) between the base pair(s) and the centromere of the chromosome, a coordinate of the base pair(s) within the genome, some other way of defining the unambiguous position of the base pair(s) within the genome, or any combination thereof.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying.” and its equivalents, may refer to a process of generating copies of a target molecule, such as a nucleic acid molecule.

As used herein, the term “hybridization.” and its equivalents, may refer to a process by which two complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

As used herein, the term “complementary.” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy.” “treatment.” and their equivalents, may refer to a composition or process that can be used to remediate a health problem. Cancer therapies (also referred to as “anticancer therapies”), for instance, include surgery, radiotherapy (e.g., a radiation therapy), chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atczolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Belcodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabomctyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib malcate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azcdra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipulcucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), tramctinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive.” and its equivalents, may refer to a type of cancer cells that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

As used herein, the terms “machine learning.” “ML.” “computer learning.” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model.” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “image,” and its equivalents, may refer to 2D or 3D array of data indicative of an array of pixels or voxels.

As used herein, the terms “transform,” “data transform,” and their equivalents, may refer to a process for converting a dataset from one domain to another domain. In various cases, transforms are reversible. Data that has been generated as a result of a transform may be referred to as “transformed data.”

As used herein, the term “domain,” and its equivalents, may refer to a set of possible inputs and/or a set of independent variables of a function or dataset. In some cases, if a dataset includes ordered pairs of first and second elements, wherein the second elements are respectively dependent on the first elements, then the domain of that dataset includes the first elements.

As used herein, the term “peak,” and its equivalents, may refer to a local or absolute minimum within a dataset or function.

As used herein, the term “trough,” and its equivalents, may refer to a local or absolute minimum within a dataset or function.

As used herein, the term “distance metric,” and its equivalents, may refer to a level of similarity between a first dataset or function and a second dataset or function.

As used herein, the term “artifact,” and its equivalents, may refer to an error in the perception or representation of information in a dataset.

As used herein, the term “filter,” and its equivalents, may refer to a system that performs one or more mathematical operations on a signal or dataset in order to reduce or enhance aspects of the signal or dataset. In some cases, a filter can be used to remove an artifact from the dataset.

### DESCRIPTION OF EXAMPLE IMPLEMENTATIONS

Various implementations of the present disclosure will now be described with reference to the accompanying Figures.

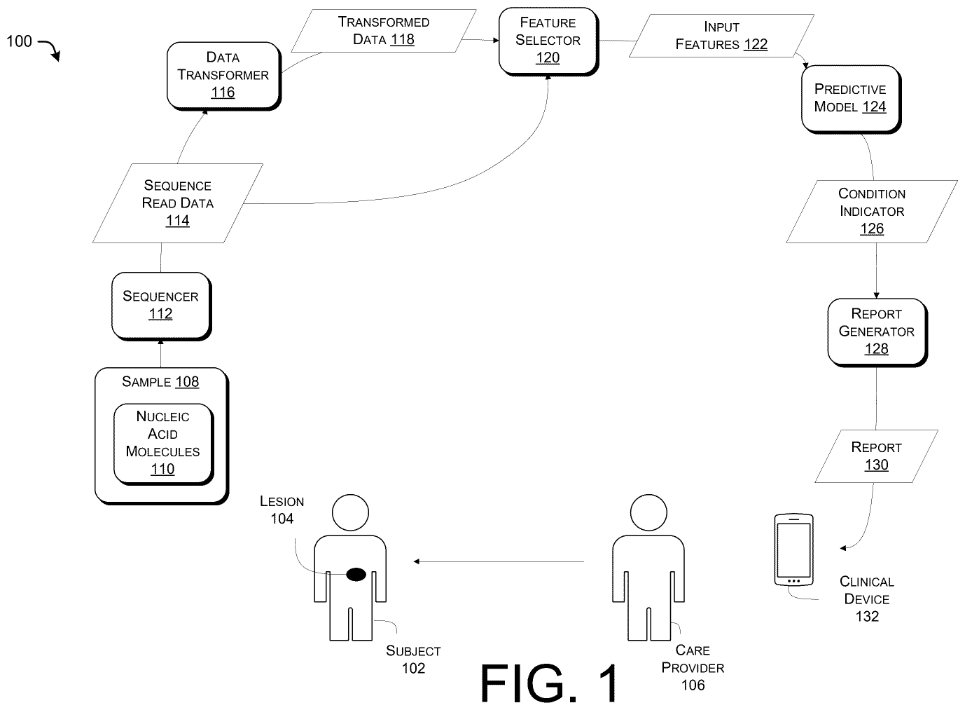

FIG. 1 illustrates an example environment 100 for predicting a condition of a subject 102 by transforming genomic information of the subject 102. In some cases, the subject 102 lacks any apparent disease or other pathological condition. For example, the subject 102 may present to a clinical environment for an assessment of a condition of the body of the subject 102, such as the general health or well-being of the subject 102.

In various implementations, the subject 102 has a disease or a suspected disease. The subject 102, for instance, may present to the clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypercosinophilic syndrome, systemic mastocytosis, familiar hypercosinophilia, chronic cosinophilic leukemia, neuroendocrine cancers, or a carcinoid tumor.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myclofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, a neurotrophic tyrosine receptor kinase (NTRK)-positive cancer, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In various cases, a care provider 106 (also referred to as a “healthcare provider”) is responsible for diagnosing and/or treating the subject 102. According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. Using the noninvasive technique, the care provider 106 may identify the presence of the lesion 104 but may be unable to determine whether the lesion 104 is a cancerous tumor using noninvasive diagnostic methodologies. In some cases in which the lesion 104 is a tumor, the care provider 106 may be unable to identify whether the tumor is metastatic or benign, or may be unable to otherwise categorize the tumor.

In various implementations, the care provider 106 is unable to accurately identify a condition of the subject 102 based solely on noninvasive diagnostic techniques. In various cases, the care provider 106 cannot conclusively determine whether the subject 102 has a type of cancer based on noninvasive diagnostic techniques. For example, the care provider 106 is unable to identify a type of the lesion 104 (e.g., a tumor) using imaging techniques. The care provider 106 may be unable to identify a characteristic of a subject presenting with a disease (e.g., cancer), wherein the characteristic is determinative of, or at least correlated with, an effectiveness of at least one therapy at treating the disease, an ineffectiveness of at least one therapy at treating the disease, a survivability (e.g., a likelihood that the subject will survive by a predetermined date or time), an expected quality of life, at least one predetermined symptom, at least one comorbidity, another factor relevant to the prognosis associated with the disease, or any combination thereof.

The care provider 106 could identify the condition of the subject 102 using histochemistry and/or immunohistochemistry. For instance, the care provider 106 could surgically remove a tissue sample from the lesion 104 and/or review the tissue sample using histochemistry and/or immunohistochemistry. However, attempting to classify the lesion 104 using these techniques has several drawbacks. First, the tissue sample may not be classifiable using conventional histological techniques, such as conventional immunohistochemical staining and review. Second, it is unlikely that the single care provider 106 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the lesion 104 was classifiable by these means, the coordinated efforts of these care providers could delay classification of the lesion 104 and could cause significant expense to the subject 102. In various examples, the delay in classification could cause significant emotional hardship to the subject 102, who could be prevented from receiving an informed prognosis for weeks. Further, the delay in classification could delay administration of a therapy to the subject 102 in order to treat the lesion 104, which could cause lasting harm to the subject 102, particularly in cases in which the lesion 104 is representative of an aggressive form of cancer.

In various implementations of the present disclosure, a condition of the subject 102 can be determined without performing histochemistry and/or immunohistochemistry. For instance, a sample 108 is obtained from the subject 102. In some examples, the sample 108 includes a tissue biopsy sample. For instance, the sample 108 is obtained by removing cells from the lesion 104 and from the subject 102. In some cases, the tissue biopsy sample is surgically excised from the subject 102. In some cases, the sample includes a liquid biopsy sample. The liquid biopsy sample 108, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 108, according to various examples, is a plasma sample obtained from the blood of the subject 102. The liquid biopsy sample 108, for instance, can be obtained in a minimally invasive procedure, which could be performed by a medical technician rather than a surgeon.

The sample 108 includes nucleic acid molecules 110. According to some examples, the nucleic acid molecules 110 include genomic DNA (gDNA). For instance, the nucleic acid molecules 110 include chromosomal DNA that is located in, or extracted from, cells in the sample 108. According to some cases, the DNA is extracted from nuclei and the cells in the sample 108 using mechanical shearing and/or the introduction of a chemical (e.g., a detergent). The DNA may be subsequently isolated from proteins and other cellular materials. In some implementations, the nucleic acid molecules 110 indicate an entire genome of the subject 102 and/or the lesion 104. Thus, a genome of the subject 102 and/or the lesion 104 can be determined by sequencing the DNA in the nucleic acid molecules 110.

In some examples, the nucleic acid molecules 110 include RNA. In some implementations, the nucleic acid molecules 110 include messenger RNA (mRNA), microRNA, non-coding RNA, functional RNA, or any combination thereof. Various RNA in the nucleic acid molecules 110 may be indicative of proteins expressed in the cells of the subject 102 and/or the lesion 104.

In some cases, the sample 108 includes cell-free DNA (cfDNA). In examples in which the subject 102 has cancer (e.g., the lesion 104 is a cancerous tumor), the cfDNA, for instance, includes circulating tumor DNA (ctDNA) and/or non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally release non-ctDNA into the bloodstream of the subject. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long.

In various cases, the sample 108 is transported to a location that is remote from the subject 102 for further processing. For example, the sample 108 is removed from the subject 102 in a clinical environment (e.g., a hospital) and is then transported to a remote laboratory for further testing and analysis.

A sequencer 112 is configured to generate sequence read data 114 indicating the sequences of the nucleic acid molecules 110. The sequencer 112, for instance, includes one or more devices that are configured to generate the sequence read data 114 by processing at least a portion of the sample 108. In some cases, the nucleic acid molecules 110 are extracted from the sample 108. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the nucleic acid molecules 110 in order to prepare the nucleic acid molecules 110 for sequencing. For instance, the sequencer 112 may ligate adapters onto the nucleic acid molecules 110 and/or amplify the nucleic acid molecules 110, such that numerous copies of the ligated nucleic acid molecules 110 are available for sequencing. Examples of the adapters include, for example, amplification primers, flow cell adapter sequences, substrate adapter sequences, or sample index sequences. The nucleic acid molecules 110 (e.g., the ligated nucleic acid molecules 110) may be amplified by generating multiple copies of the nucleic acid molecules 110 using one or more techniques such as polymerase chain reaction (PCR), a non-PCR amplification technique, or an isothermal amplification technique.

The sequencer 112 may identify the length, position, and identity of the bases in the nucleic acid molecules 110 by sequencing the nucleic acid molecules 110 (e.g., the amplified and/or ligated nucleic acid molecules 110). In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the nucleic acid molecules 110 fragments obtained from the sample 108. In some examples, the sequencer 112 is configured to perform targeted sequencing. For instance, the sequencer 112 may determine whether the nucleic acid molecules 110 fragments contain one or more predetermined sequences at one or more genomic locations.

In various cases, the sequencer 112 includes one or more sensors that are configured to detect physical signals (also referred to as “detection signals”) that are indicative of the nucleotide sequences of the nucleic acid molecules 110. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated nucleic acid molecules 110 as templates. The optical signals detected by the optical sensor(s), for instance, are indicative of the sequences of the nucleic acid molecules 110. The sequencer 112 may perform nanopore sequencing. In various cases, the sequencer 112 includes one or more electrical sensors configured to measure an electrical signal (e.g., an electrical current) across a substrate as the ligated nucleic acid molecules 110 are directed through a nanopore extending through the substrate. The electrical signal over time, in various cases, is indicative of the sequences of the nucleic acid molecules 110 in the sample 108. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some implementations, the sequencer 112 performs RNA sequencing (RNA-seq) on the nucleic acid molecules 110. For example, the nucleic acid molecules 110 include RNA that is extracted from the sample 108. In some examples, the RNA in the nucleic acid molecules 110 is fragmented. In various implementations, complementary DNA (cDNA) is generated using reverse transcriptase, such that the cDNA includes sequences that are complementary to the RNA in the nucleic acid molecules 110 from the sample 108. The cDNA, according to various cases, can be sequenced using the DNA sequencing techniques described above. Accordingly, in some cases, the sequence read data 114 indicates sequences of RNA present in the sample 108, which may be indicative of the transcriptome of the subject 102 and/or the lesion 104.

In various cases, the sequencer 112 performs sequencing on a subset of the nucleic acid molecules 110. For instance, the sequencer 112 may perform targeted sequencing on one or more predetermined genes, such as any of the genes described herein. The sequencer 112, in some cases, may refrain from sequencing at least a portion of the nucleic acid molecules 110 that do not correspond to the subset.

The sequence read data 114, according to various instances, is in a spatial domain. For example, the sequence read data 114 may be indicative of the genomic locations of the nucleic acid molecules 110 in the sample 108. In various cases, the sequence read data 114 may be difficult to analyze directly. Although it may be possible to identify, in the sequence read data 114, attributes or other characteristics that are predictive of the condition of the subject 102, such analyses may utilize numerous computing resources.

In various implementations of the present disclosure, the sequence read data 114 is output to a data transformer 116 rather than analyzed directly. The data transformer 116 is configured to generate transformed data 118 by transforming the sequence read data 114 from a first domain (e.g., the spatial domain) to a second domain that is different than the first domain. That is, the second domain is an “alternate” domain to the first domain. In some cases, the transformed data 118 includes data representing the sequence read data 114 in the second domain. In some examples, the transformed data 118 includes one or more images representing the sequence read data 114 in the second domain.

Various types of transformations can be performed by the data transformer 116. In some examples, the data transformer 116 is configured to generate the transformed data 118 by performing a Fourier transform on the sequence read data 114. The transformed data 118, for instance, is in a frequency domain. According to some examples, the data transformer 116 is configured to perform a Fast Fourier Transform (FFT) on the sequence read data 114. In some cases, the data transformer 116 is configured to perform a continuous Fourier transform on a function representative of the sequence read data 114. In various examples, the data transformer 116 is configured to perform a discrete Fourier transform (DFT) on the sequence read data 114. According to some cases, the data transformer 116 is configured to perform a short-time Fourier transform (STFT) on the sequence read data 114.

In some examples, the data transformer 116 is configured to generate the transformed data 118 using one or more other types of transforms. For example, the data transformer 116 may generate the transformed data 118 by performing a Hartley transform, a Laplace transform, a Mellin transform, a wavelet transform (e.g., a continuous wavelet transform (CWT), a discrete wavelet transform (DWT), a fast wavelet transform (FWT), a complex wavelet transform, a Newland transform, a stationary wavelet transform (SWT), a second generation wavelet transform (SGWT), a dual-tree complex wavelet transform (DTCWT), etc.), or any combination thereof, on the sequence read data 114. In some cases, the data transformer 116 generates the transformed data 118 by generating a Taylor series or Taylor expansion of the sequence read data 114. Example transforms are described, for instance, in Farge, Annu. Rev. Fluid Mech. 24:395-457 (1992), which is incorporated by reference herein its entirety.

According to various cases, the transformed data 118 represents at least one locus of interest indicated by the sequence read data 114. For instance, the transformed data 118 may include a second-domain mapping of a portion of the sequence read data 114 that reflects at least one gene-of-interest of the subject 102 and/or the lesion 104, as reflected in the sequence read data 114. Examples of genes with potential relevance to a determination of whether the subject 102 has a type or subtype of cancer include ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRF11, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703, In some cases, the genes include at least one estrogen receptor (ER) gene and/or at least one progesterone receptor (PR) gene. In some cases, the genes include one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB, In some examples, the genes include one or more of TP53, CTNNNB1, L1CAM, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1.

In some cases, characteristics of the sequence read data 114 can be more efficiently identified by transforming the sequence read data 114 into an alternate domain. Accordingly, transforming the sequence read data 114, in some examples, can greatly reduce the amount of processing resources utilized to identify the condition of the subject 102. Further, in some cases, transforming the sequence read data 114 enables new characteristics to be identified using the sequence read data 114.

A feature selector 120 identifies input features 122 of the nucleic acid molecules 110 by analyzing the transformed data 118. In various implementations, the feature selector 120 identifies, calculates, or otherwise determines the input features 122 based on the sequences of the nucleic acid molecules 110 indicated in the transformed data 118. One or more types of features are identified by the feature selector 120. In various implementations, the input features 122 are genomic features (also referred to as “genomic features”). That is, the input features 122 may be derived from the sequence read data 114 in addition to the transformed data 118.

In some examples, the input features 122 include at least one distance metric. For example, the feature selector 120 may generate the distance metric by comparing the transformed data 118 to pre-classified data that is in the same domain as the transformed data 118. In some cases, the pre-classified data is generated based on nucleic acid molecules obtained from one or more individuals with known conditions and/or subtypes of conditions. For example, the pre-classified data may include transformed data of an individual with a known type of cancer (e.g., bladder cancer) or a known cancer subtype (e.g., urothelial carcinoma). According to some cases, the pre-classified data is generated based on nucleic acid molecules obtained from one or more individuals with the absence of a particular condition, such as an individual without cancer. In various cases, the distance metric(s) may represent a similarity between the transformed data 118 and the pre-classified data. For example, the distance metric(s) may be generated by cross-correlating and/or convolving the transformed data 118 and the pre-classified data. In some cases, the distance metric(s) include the value of a peak and/or mean of the cross-correlated and/or convolved data. According to various implementations, a magnitude of the distance metric(s) is indicative of a likelihood that the nucleic acid molecules 110 of the subject 102 reflect the known condition or subtype of the pre-classified data. Thus, the condition of the subject 102 can be identified using the distance metric(s).

According to some implementations, the feature selector 120 performs image processing techniques in order to generate the input features 122. In some cases, the feature selector 120 generates a digital image based on the transformed data 118. For example, the feature selector 120 may generate a spectrogram or other graphical representation of the transformed data 118. In some cases, the feature selector 120 generates the input features 122 by analyzing the image of the transformed data 118.

In some cases, the feature selector 120 includes a machine learning (ML) model configured to identify features of the image that are predictive of the condition of the subject 102. For instance, the feature selector 120 may include a convolutional neural network (CNN) that generates the input features 122 in response to receiving the image representative of the transformed data 118. According to various examples, the CNN may include multiple blocks and/or layers that are each defined by a kernel (e.g., a digital image filter). Each block and/or layer may be configured to convolve and/or cross-correlate the kernel with pixels of an input image, thereby generating an output image. In some cases, the blocks and/or layers are arranged in series, such that the input image of one block and/or layer may be the output image of another block and/or layer. Each block and/or layer may further be defined according to a receptive field of its kernel and/or a stride size of the kernel.

In some examples, the CNN of the feature selector 120 is pretrained. For example, the values of the kernel of each block and/or layer may be optimized based on training data prior to receiving the image of the transformed data 118. In some examples, the training data includes other images of other transformed data, as well as manually obtained indications of the types of input features that the CNN is being trained to identify. The CNN, for instance, may be trained using a supervised learning technique. Because the CNN is pretrained, the CNN may be configured to output the input features 122 in response to receiving the image of the transformed data 118.

According to some examples, the feature selector 120 is configured to filter the transformed data 118. For instance, the feature selector 120 may be configured to apply one or more filters in the domain of the transformed data 118. For example, the feature selector 120 may apply a filter by convolving, cross-correlating, or multiplying the second-domain representation of the filter with the transformed data 118. By filtering the transformed data 118, in some cases, the feature selector 120 can reduce or eliminate artifact in the transformed data 118 and/or enhance one or more characteristics indicative of the input features 122 in the transformed data 118. In some cases, it may be more computationally efficient to apply the filter to the transformed data 118 in the second domain than to the sequence read data 114 in the first domain. Examples of filters include a Butterworth filter, a Chebyshev filter, a finite impulse response (FIR) filter, or an infinite impulse response (IIR) filter. In some cases, the filter applied by the feature selector 120 is a low-pass filter, a high-pass filter, or a bandpass filter. For instance, the filter may be defined by one or more cutoff frequencies.

One or more types of features may be included in the input features 122. In some cases, the input features 122 are derived exclusively by the feature selector 120 based on the transformed data 118. For example, the input features 122 may include a digital image of at least a portion of the transformed data 118 and/or features derived based on the digital image. In some cases, the input features 122 include at least one peak of the transformed data 118, at least one trough of the transformed data 118, a distance metric associated with the transformed data 118, an indication of whether at least a portion of the transformed data 118 exceeds a threshold, or any combination thereof. In particular examples, the input features 122 are derived by the feature selector 120 based on a combination of the transformed data 118 and the sequence read data 114.

In some cases, the input features 122 include a mismatch repair deficiency (MMRD) probability score. In various cases, the MMRD probability score indicates a likelihood that one or more MMR pathways of cells in the sample 108 are ineffective at performing mismatch repair. In some implementations, the MMRD probability score is determined by determining genomic features by analyzing the sequence read data 114, inputting the genomic features into at least one trained machine learning model trained to generate the MMRD probability score based on previously analyzed data from a population omitting the subject 102. The genomic features relevant to the MMRD probability score include, for instance, a fraction unstable score, a composite COSMIC single-base substitution signature, a COSMIC indel signature, a copy number signature, a tumor mutational burden score, a blood-based tumor mutational burden score, a germline status for a mutation in one or more genes associated with DNA mismatch repair (MMR) (also referred to as “MMR genes”), a methylation status for the one or more MMR genes, a methylation status for one or more promoters associated with the one or more MMR genes, a methylation status of one or more enhancers associated with the one or more MMR genes, or any combination thereof. Examples of the MMR genes include, for instance, MSH2, MSH6, PMS2, or MLH1.

The input features 122, in some examples, include a copy number state of one or more genetic loci indicated by the sequence read data 114. In various implementations, a number of copies of a predetermined sequence at a given locus in the genome of the subject 102 and/or the lesion 104 (also referred to as a “copy number” of the locus) is determined. The copy number state, in various implementations, may indicate copy numbers of one or more loci in the genome of the subject 102 and/or the lesion 104. For instance, the copy number state may indicate the presence and/or amount of copies of various sequences present in the genome of the subject 102 and/or the lesion 104, which may be due to copy number variation.

According to various examples, the sequence read data 114 may represent a genome of the subject 102 and/or the lesion 104. Various portions of the sequence read data 114 are aligned with at least one reference sequence (e.g., a reference genome). The aligned data is segmented using at least one segmentation technique (e.g., a circular binary segmentation (CBS) method, a maximum likelihood method, a hidden Markov chain method, a walking Markov method, a Bayesian methods, a long-range correlation method, a change point method, or any combination thereof), thereby generating non-overlapping segments of the sequence read data 114, wherein a sequence associated with a given segment is associated with the same copy number (e.g., a number of instances in which the sequence appears in the segment). Various genetic loci are binned, or otherwise sorted, with respect to the segments of the genome of the subject 102 and/or the lesion 104. The copy number state, for instance, is representative of the respective copy numbers associated with the genetic loci. In some cases, the copy number state is dependent on (e.g., assigned based on) a major allele coverage ratio and a minor allele coverage ratio, as well as one or more copy number grid models.

In some implementations, the input features 122 include the presence or absence of a variant (e.g., a pathogenic variant) in one or more genes associated with classifying the lesion 104. In various cases, the genes include one or more of the genes with potential relevance to a determination of whether the subject 102 has a type or subtype of cancer, as listed above.

In some cases, the input features 122 are indicative of microsatellite instability (MSI). Microsatellites are highly polymorphic DNA-repeat regions. In certain examples. “microsatellite” refers to a repetitive nucleic acid having repeat units of less than about 10 base pairs or nucleotides in length. In certain examples, a microsatellite refers to a tract of tandemly repeated (i.e., adjacent) DNA motifs ranging from one to six or up to ten nucleotides, with each motif repeated 5 to 50 repeated times. During DNA replication, mutations (e.g., insertions or deletions) are more likely to be introduced at microsatellites than various other portions of the genome. In various cases, these mutations are corrected via MMR pathways. However, if the MMR pathways are impaired (e.g., the MMR genes of the hosting cell include variants that impede function), then the mutations at the microsatellites may be substantially retained. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol. 26 (2): c15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma. According to various examples, “MSI score” refers to an amount of instability in one or more microsatellites. For example, an MSI score can be represented as a fraction (i.e., an “MSI fraction”) of instability in the one or more microsatellites. Other types of portions of DNA may be associated with a high likelihood of mutations. In some cases, the input features 122 include a fraction unstable score, indicative of mutations in the microsatellites and other portions of the genome that are prone to mutations.

In various cases, an MSI score can be determined based on a predetermined set of repetitive loci (e.g., 2000 repetitive loci, each with a minimum of 5 repeat units of mono-, di-, and trinucleotides). By evaluating the sequence read data 114, the feature selector 120 may determine lengths of repetitive sequences corresponding to the loci. If an example locus among the loci corresponds to a predetermined repeat length, the locus is considered to be “unstable.” The MSI score, for instance, is determined by determining an amount of the unstable loci (e.g., a fraction of the unstable loci with respect to the total number of repetitive loci evaluated). In some cases, the MSI score is used to determine whether the subject 102 and/or lesion 104 is MSI-High (MSI-H). For example, MSI-H status may be applicable if the MSI score is greater than a threshold (e.g., 0.5%). Techniques for determining MSI scores are described, for instance, in Woodhouse et al., “Clinical and analytical validation of FoundationOne LiquidCDx, a novel 324-Gene cfDNA-based comprehensive genomic profiling assay for cancers of solid tumor origin,” PLOS ONE 15 (9) (2020).

In some implementations, the input features 122 include a mutation signature. In various cases, a mutational signature can represent an amount and/or identity of mutations (e.g., insertions, deletions, double-base substitutions, single-base substitutions, or any combination thereof) indicated in the nucleic acid molecules 110 from the subject 102. In some cases, the mutational signature indicates an amount (e.g., number or percentage) of individual classes of base substitutions present in the nucleic acid molecules 110. For instance, the classes include single-base substitutions including C>A, C>G, C>T. T>A, T>C, and T>G.

A mutational signature can be derived by comparing the sequences indicated in the sequence read data 114 to at least one reference sequence, such as a reference genome. For example, the input features 122 may include a Catalogue Of Somatic Mutations In Cancer (COSMIC) mutational signature, such as a COSMIC indel signature. In some cases, the input features 122 include a single-base substitution signature.

In various examples, the input features 122 include a tumor mutational burden (TMB) score. Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation. In certain examples, “tumor mutational burden” or “TMB score” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In addition, germline variants do not reflect the biology of somatic mutation for the purposes of TMB determinations. In various cases, driver mutations are excluded from a TMB calculation.

In some cases, the input features 122 include the presence, amount, type, or any combination thereof, of one or more hotspot mutations. Hotspots, for instance, can refer to loci in the genome of the subject 102 and/or the lesion 104 that are prone to mutation. Examples of hotspots include CpG islands, microsatellites, centromeric DNA, telomers, subtelomeric regions, common fragile sites, palindromic AT-rich repeats (PATRRs), G-quadruplexes, R-loops, and the like.

Hotspot mutations give rise to oncological outcomes. PhyloP, SIFT, Grantham. COSMIC and PolyPhen-2 are in silico tools that can be used to assess pathogenicity of identified variants. Exemplary hotspot genes and mutations include EGFR exon 19 activating mutation. EGFR exon 19 deletion. EGFR exon 19 insertion, EGFR exon 19 sensitizing mutation, EGFR exon 20 activation mutation. EGFR exon 20 insertion. EGFR G719 mutation, EGFR L858R mutation, EGFR L861 mutation. EGFR S768 mutation, EGFR T790M mutation, C797 mutation, KIT activating mutation, KRAS activating mutation, MET activating mutation, NRAS activating mutation. PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

The input features 122, in particular examples, include the presence, amount, type, or any combination thereof, of one or more aneuploidy events. For instance, the input features 122 may indicate whether the subject 102 and/or the lesion 104 includes one or more extra chromosomes (e.g., greater than a pair of 23 chromosomes) or one or more missing chromosomes (e.g., less than the pair of 23 chromosomes).

In some implementations, the input features 122 include a tumor purity of the sample 108. In various implementations, the tumor purity represents an amount of the nucleic acid molecules 110 that originate from a tumor (e.g., the lesion 104) with respect to a total amount of the nucleic acid molecules 110 in the sample 108. Tumor purity can be estimated, for instance, based on a presence or amount of somatic copy-number alterations (SCNA), single-nucleotide variants (SNVs), minor allele frequency (MAF), or any combination thereof, observed with respect to the sequence read data 114.

In some cases, the input features 122 include additional biomarker data. That is, the input features 122 may include non-genomic features. For instance, input features 122 may include data indicating at least one of a histological and/or immunohistological image of the sample 108 or another sample of the lesion 104, a genomic alteration, or a viral status of the subject 102 and/or lesion 104. The additional biomarker data may be generated based on the sample 108, medical images, or other samples obtained from the subject 102. In some cases, the additional biomarker data includes an image of a stained section of the lesion 104. For instance, the stained section is stained with hematoxylin and eosin (H&E) and/or at least one immunostain.

To categorize the cancer, a predictive model 124 is configured to generate a condition indicator 126 based on the input features 122. The predictive model 124, for example, may include one or more mathematical and/or computer-based models that are configured to predict the condition indicator 126 based on the input features 122. For instance, the predictive model 124 may include a regression model, threshold rule, confidence interval, or other type of statistical model capable of categorizing the cancer based on the input features 122. In various cases, the predictive model 124 includes at least one classifier configured to generate the condition indicator 126 based on the input features 122.

In various implementations, the predictive model 124 includes at least one trained ML model configured to output the condition indicator 126 in response to receiving the input features 122 in input data. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique, wherein the training data further included ground truth prognostic classifications of the individuals in the population, such that the parameters were optimized to minimize a loss between predicted prognostic classifications generated by the ML model(s) based on the features of the population and the ground truth prognostic classifications of the cancers experienced by the individuals in the population. To increase training robustness, the population represented by the training data may include individuals without cancer, as well as individuals with a variety of cancer types and metastasis states. Various types of ML models can be included in the predictive model 124, such as a neural network (e.g., a CNN, which may be different than a CNN in the feature selector 120), a nearest-neighbor model, a regression analysis model, a clustering model, a principal component analysis model, a gradient boosting model, a random forest, or any combination thereof. In some cases, the predictive model 124 includes a hybrid model, that includes multiple types of ML models. For instance, the predictive model may include a CNN and a clustering model.

In particular examples, the predictive model 124 includes a clustering model. In various implementations, the clustering model is pre-trained based on training data that includes population features. According to various implementations, the population features include genomic features and/or additional biomarker data of the population. In some cases, the population features further include one or more known prognostic features and/or prognostic classifications of the population. In various implementations, at least one computing device is configured to cluster the population features. The clustering model, for instance, stores, includes, or otherwise indicates the determined clusters.

In various examples, the population characteristics are defined in a multi-dimensional feature space. In various cases, the feature space has n dimensions (e.g., a dimensionality value of n), wherein n corresponds to the number of feature types included in the population feature. For example, one dimension may correspond to a number of peaks in the transformed data 118 that exceed a threshold, another dimension may refer to a distance metric representing a similarity between the transformed data 118 and pre-classified transformed data based on a sample obtained from an individual with a particular type of cancer, and so on. In various cases, data objects representing the population features of the population are plotted or otherwise defined in the feature space. In some examples in which n is greater than two, the data objects are projected onto an m-dimensional feature space using multi-dimensional scaling, wherein m is between 1 and n−1 (inclusive). Multi-dimensional scaling can be achieved using various techniques. For instance, multi-dimensional scaling can be performed using at least one of a statistical method (e.g., t-distributed stochastic neighbor embedding (t-SNE), uniform manifold approximation and projection (UMAP), representation learning (e.g. principal component analysis (PCA), independent component analysis (ICA), etc.), ML-based latent space learning (e.g. autoencoders, transformers, generative adversarial networks, etc.). Accordingly, in some cases, the data objects can be visualized in a Cartesian coordinate system.

Within the feature space (whether it has two or more than two dimensions), the data objects are separated from each other by distances. Various types of distances can be utilized in implementations of the present disclosure. For example, the distances may include Euclidian distances, Manhattan distances, Hamming distances, Minkowski distances, Chebyshev distances, or any combination thereof.

Various clustering techniques can be utilized to generate the clustering model. For instance, the clusters may be generated using k-means clustering, density-based clustering, centroid-based clustering, spectral clustering, distribution-based clustering, hierarchical clustering, or any combination thereof. In some implementations, the clustering model is generated by performing hierarchal clustering on the data objects representing the population features. In various cases, the clusters include two or more data objects that are within proximity of each other (e.g., within a predetermined distance of one another) in the feature space. For instance, a cluster may include two or more data objects that are within a predetermined distance (e.g., Euclidian distance) of one another in the feature space. In some implementations, a data object is included in a cluster if the data object is within an appropriate distance of a linkage criterion representing one or more data objects that are already defined within the cluster. Various implementations of the present disclosure utilize one or more linkage criteria, such as a single-linkage criterion, a complete-linkage criterion, an average-linkage criterion (e.g., a weighted average criterion, an unweighted average criterion), a centroid-linkage criterion, a median linkage criterion, a Ward linkage criterion, a minimum error sum of squares criterion, a min-max criterion, a Hausdorff linkage criterion, a medoid linkage criterion, a minimum energy clustering criterion, or any combination thereof.

In some cases, agglomerative clustering is used to generate the clusters. For example, initially, each data object is defined within the feature space without clustering. Subsequently, pairs of adjacent data objects may be clustered together. In some examples, the process of generating a cluster based on independent data objects in a feature space, or of adding a data object to an existing cluster, may be referred to as “merging.”

In some examples, divisive clustering is used to generate the clusters. For example, the data objects may be defined into a single cluster in the feature space. Subsequently, the single cluster may be divided into multiple clusters. In some instances, the process of dividing a preliminary cluster into multiple subsequent clusters, or of removing a data object from a cluster, may be referred to as “splitting.”

In various cases, each cluster is defined according to a boundary (also referred to as a “border”). In some implementations, data objects outside of the boundary of a cluster are not part of the cluster. Data objects inside of the boundary of the cluster are part of the cluster. Depending on the data objects, the linkage criterion, the feature space, and other characteristics of the training data, the clusters may have irregular shapes within the feature space. In various cases, the clustering model includes the boundaries of the clusters generated based on the data objects defined by the population features.

According to various cases, each cluster in the clustering model is associated with one or more conditions. In some cases, at least one condition is defined in at least one dimension of the feature space, such that the clusters are defined according to the prognostic feature(s). In some examples, the population features used to define the clusters omit one or more conditions. Once the clusters are generated based on non-condition features (e.g., genomic features and/or additional biomarker data), conditions associated with the clusters are subsequently determined. For example, an example cluster may be defined based on the data objects representing the non-condition population features of m members of the population, wherein m is an integer that is greater than one. In various cases, conditions of the m members of the population are determined. Common conditions of the population (e.g., cancer status) are determined. For example, if greater than a threshold number of the m members have a cancer that is resistant to a predetermined therapy, than resistance to the predetermined therapy may be associated with the example cluster. In various cases, each cluster may be labeled with, or otherwise associated with, one or more conditions. The one or more prognostic features associated with a given cluster form the condition associated with the cluster. In various cases, each cluster in the clustering model is associated with a condition.

In various implementations, the cancer of the subject 102 is categorized by comparing the input features 122 of the subject 102 to the clusters in the clustering model. The condition indicator 126 is determined based on a comparison between the input features 122 and the clusters in the clustering model. In various cases, a data object defined by the input features 122 of the subject 102 is defined in the feature space of the clustering model. The clustering model, for instance, may determine that the data object is present within the boundary of a particular cluster that was previously defined based on the training data. In some cases, the clustering model determines that the data object is associated with a particular cluster based on a distance between the data object and the particular cluster in the feature space. In some cases, the distance is at least one of a Euclidian distance, a Manhattan distance, a Hamming distance, a Minkowski distance, a Chebyshev distance, or any combination thereof. For instance, the clustering model determines that the distance between the data object and the boundary and/or a centroid of the particular cluster is below a threshold distance. In some examples, the clustering model classifies the condition of the subject 102 into a classification associated with the particular cluster by determining that a distance between at least one data object corresponding to the population features in the cluster is below a threshold distance.

In various cases, the condition indicator 126 of the sample 108 is generated. For example, the clustering model may determine that the subject 102 is associated with one or more prognostic features associated with the cluster in which the input features 122 belong. In various examples, the prognostic features may include a predicted disease of the subject 102, predicted characteristics of the disease that is experienced by the subject 102, predicted symptoms (e.g., predicted chronic symptoms, such as heart disease, diabetes, high blood pressure, etc., or predicted medical events, such as heart attack, stroke, pre-eclampsia, etc.) of the subject 102, predicted causes of the disease, or the like. For instance, the condition indicator 126 includes one or more of a predicted condition (e.g., disease) of the subject 102; a predicted disease subtype of the subject 102; a predicted survivability of the subject 102; one or more predicted symptoms of the subject 102; a predicted (e.g., suggested) effective therapy to treat the predicted disease of the subject 102; a dosage of one or more therapeutic agents (e.g., biologics, chemotherapeutic agents, etc.) predicted to treat the condition of the subject 102, a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted diabetes status of the subject 102; a predicted body mass index (BMI) of the subject 102; a predicted smoking history of the subject 102; a predicted breast density of the subject 102; a clinical trial that the subject 102 is predicted to qualify (e.g., be eligible) for; or a characteristic of the predicted disease of the subject. Accordingly, the condition of the subject 102 can be determined based on the input features 122.

In some implementations, the predictive model 124 is unable to conclusively categorize the condition of the subject 102. For example, the predictive model 124 may determine that the input features 122 of the subject 102 do not fit within any of the previously defined clusters in the clustering model. In various cases, the predictive model 124 may output an indication that that the categorization of the condition is inconclusive.

A report generator 128 is configured to generate a report 130 based, at least in part, on the condition indicator 126. The report 130, for example, includes consumable data that can inform the care provider 106 about the predicted condition of the subject 102. In various implementations, the report 130 may indicate the results of additional analyses, such as the results of a histological study, whole transcriptome sequencing, cfRNA sequencing, whole exome sequencing, whole genome sequencing, a cancer (e.g., DNA) hotspot panel test, a DNA methylation test, a TMB test, a DNA fragmentation test, an RNA fragmentation test, a microsatellite instability (MSI) test, or a viral status test. The performance of such tests is within the ordinary skill of the art, with additional detail provided elsewhere herein. The report 130, for example, may include a genomic profile of the subject 102 based on various combinations of the above analyses and tests.

In some implementations, the report 130 indicates that a follow-up test of the subject 102 is indicated. For instance, in response to determining that the categorization of the condition of the subject 102 is inconclusive, the report generator 128 may generate the report 130 to indicate that one or more additional tests (e.g., a histological study, genome sequencing, exome sequencing, additional DNA sequencing, RNA sequencing, transcriptome sequencing, etc.) should be performed in order to accurately identify the condition of the subject 102.

In various cases, the report 130 is output to a clinical device 132. For example, the report generator 128 transmits the report 130 to the clinical device 132. In various implementations, the clinical device 132 is a computing device that is operated by, owned by, or otherwise associated with the care provider 106.

For instance, the clinical device 132 may be a desktop computer, a laptop computer, a smart phone, or some other computing device associated with the care provider 106. The clinical device 132, in various cases, outputs the report 130 to the care provider 106. In some cases, the clinical device 132 includes a display (e.g., a screen) that visually presents the report 130. In various cases, the clinical device 132 includes a speaker that outputs a sound indicative of the report 130. The clinical device 132, in various cases, may output the information in the report 130 using one or more output mechanisms or devices.

The care provider 106 may review the report 130 by interacting with the clinical device 132. The report 130, in various cases, may enhance the clinical decision-making of the care provider 106. For instance, the care provider 106 may prepare and/or administer a therapy to the subject 102 based on the report 130. According to various implementations, the care provider 106 may initiate the therapy and/or refer the subject 102 to another care provider to receive the therapy. In various cases, if the predicted condition of the subject 102 is a disease (e.g., cancer), the care provider 106 may prescribe, recommend, or administer an agent in order to treat the disease the subject 102.

In various implementations, the care provider 106 may develop a diagnosis and/or prognosis of the subject 102 based on the report 130. In various implementations, the care provider 106 may communicate information in the report 130 to the subject 102.

FIG. 1 illustrates various elements that can be embodied in one or more computing devices. For example, at least a portion of the functions of one or more of the sequencer 112, the data transformer 116, the feature selector 120, the predictive model 124, the report generator 128, or the clinical device 132 are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, one or more of the sequence read data 114, the transformed data 118, the input features 122, the condition indicator 126, or the report 130, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3rd Generation Partnership Project (3GPP) radio network, such as a Long Term Evolution (LTE) network, a New Radio (NR) network; or a cellular core network such as a 3rd Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to the care provider with unexplained weight loss and fatigue. The care provider 106 may, without ordering imaging of the subject 102, obtain the sample 108 from the blood of the subject 102. The sequencer 112 may generate sequence read data 114 based on DNA fragments within the blood sample 108 of the subject 102. For example, the sequence read data 114 may represent positions of the endpoints of the DNA fragments within one or more genes associated with cancer. A data transformer 116, in various cases, generates the transformed data by performing a Fourier transform (e.g., an FFT) on the sequence read data 114. The feature selector 120, in various cases, identifies one or more peaks in the transformed data 118 using peak detection. The input features 122 includes indications of the peak(s) in the transformed data 118. The peak(s) in the transformed data 118 are indicative of whether the subject 102 has prostate cancer. For example, the predictive model 124 is trained to determine whether the subject 102 has prostate cancer by analyzing the peak(s). For instance, the predictive model 124 includes at least one ML model trained to identify a likelihood that the subject 102 has prostate cancer based on the peak(s). Upon analyzing the peak(s) in the input features 122, the predictive model 124 outputs the condition indicator 126 that indicates the subject 102 has a 98% likelihood of having prostate cancer. That indication is summarized on the report 130 and output to the clinical device 132. Thus, the care provider 106 may inform the subject 102 of the likely diagnosis and begin discussions of treatments without performing invasive testing on the subject 102.

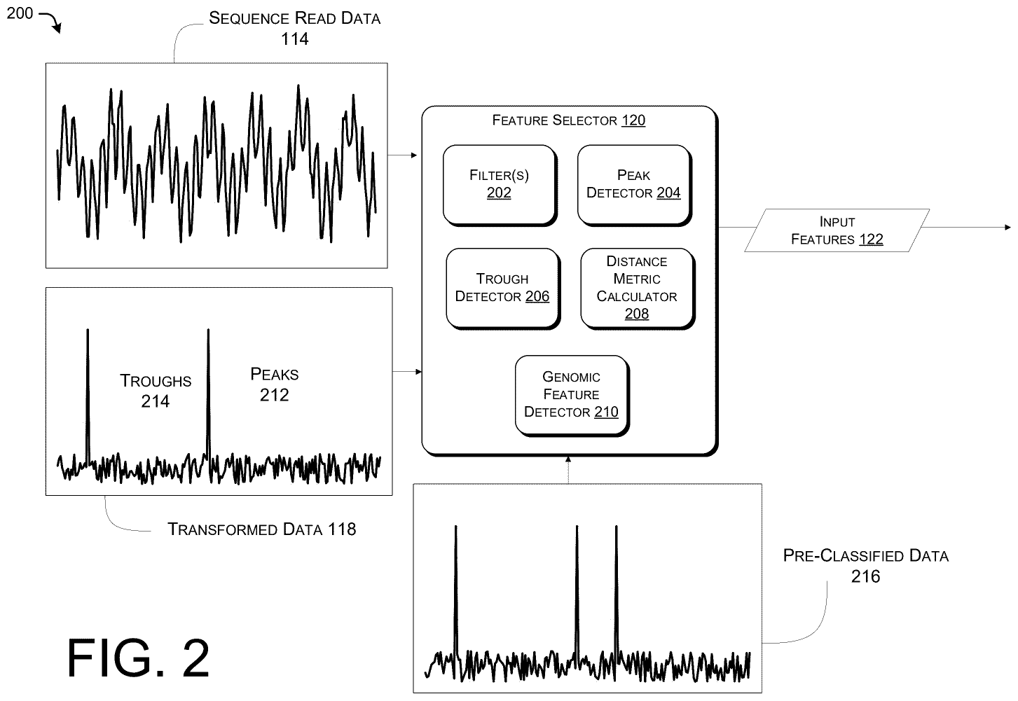

FIG. 2 illustrates example signaling 200 for selecting features for classifying a condition of a subject based on transformed genomic information of the subject. The signaling 200 is to and from the feature selector 120 described above with reference to FIG. 1, for instance. The signaling 200 further includes the sequence read data 114, the transformed data 118, and the input features 122 described above with reference to FIG. 1.

The sequence read data 114 represents sequences of nucleic acid molecules in a sample obtained from a subject. In some examples, the sequence read data 114 is multi-dimensional data. One of the dimensions of the sequence read data 114, for instance, represents genomic position. In some examples, one of the dimensions of the sequence read data 114 represents a number of endpoints (e.g., a number of right endpoints and/or left endpoints, also referred to as “endpoint counts”) of fragments in the nucleic acid molecules detected in the sample. In some examples, the dimensions of the sequence read data 114 include at least one of a presence (or absence) of variants in the nucleic acid molecules, an amount of signal observed by a sequencer (e.g., at a given genomic position) from the nucleic acid molecules, a read depth, a length of fragments in the nucleic acid molecules, or any combination thereof. The sequence read data 114, for instance, represents the sequences of the nucleic acid molecules in a spatial domain that is defined by genomic position. In some cases, the sequence read data 114 represents genomic positions in at least one locus. For instance, the sequence read data 114 may be limited to genomic positions in one or more genes-of-interest that are relevant for classifying the condition of the subject.

Similar to the sequence read data 114, the transformed data 118 is multi-dimensional and also represents the sequences of the nucleic acid molecules in the sample obtained from the subject. However, the transformed data 118 may be mapped to an alternate domain compared to the spatial domain of the sequence read data 114. For instance, a dimension of the sequence read data 114 may be a frequency domain rather than a spatial domain. The transformed data 118 may be generated by performing at least one transform on the sequence read data 114. Examples of transforms include a Fourier transform, a Laplace transform, a Mellin transform, a wavelet transform (e.g., a continuous wavelet transform (CWT), a discrete wavelet transform (DWT), a fast wavelet transform (FWT), a complex wavelet transform, a Newland transform, a stationary wavelet transform (SWT), a second generation wavelet transform (SGWT), a dual-tree complex wavelet transform (DTCWT), etc.), or any combination thereof.

In various cases, the feature selector 120 generates the input features 122 based on the sequence read data 114 and the transformed data 118. The input features 122, for instance, include characteristics of the subject that are relevant to determining a condition of the subject, and which are derived based on the sequence read data 114 and/or the transformed data 118.

In some examples, the feature selector 120 includes at least one filter 202 configured to remove and/or enhance characteristics of the sequence read data 114 and/or the transformed data 118. In particular cases, the filter(s) 202 is configured to remove an artifact of the sequence read data 114 and/or the transformed data 118. Examples of filters that can be included in the filter(s) 202 include at least one of a Butterworth filter, a Chebyshev filter, an FIR filter, an IIR filter, a low-pass filter, a high-pass filter, or a bandpass filter. In some cases, the filter(s) 202 is a set of data having a shape that is suitable for removing and/or enhancing characteristics of the sequence read data 114 and/or the transformed data 118. The filter(s) 202, for instance, is multiplied, convolved, or cross-correlated with the sequence read data 114 and/or the transformed data 118. In some cases in which the sequence read data 114 is in a spatial domain and the transformed data 118 is in a frequency domain, the filter(s) 202 is convolved with the sequence read data 114 and/or multiplied with the transformed data 118. In some cases, the filter(s) 202 is applied to the transformed data 118, and a reverse transform is performed on the filtered transformed data 118 in order to obtain filtered sequence read data 114. According to some examples, the filtered sequence read data 114 is utilized to perform various functions described herein.

In various cases in which the transformed data 118 is in a frequency (or frequency-related) domain, the transformed data 118 may include low-frequency and/or high-frequency artifact. Examples of low-frequency artifact include copy number deletions and/or copy number amplifications, when those features have limited to no relevance to the condition of the subject that is being assessed. In some cases, the sequencing technique used to generate the sequence read data 114 utilizes bait molecules associated with particular genomic regions (e.g., loci) of interest. Due to the physical limitations of this sequencing technique, there may an observed signal decay in genomic positions within a threshold of the bait molecules and/or at edges of the genomic regions of interest. This signal decay is another example of potential low-frequency artifact. In some examples, the filter(s) 202 includes a band-pass and/or a high-pass filter with a cutoff frequency that is suitable for removing one or more types of low-frequency artifact. In some cases, the sequence read data 114 further includes one or more types of high-frequency artifact. For example, the high-frequency artifact may include misreads during sequencing, base-level sequencing errors, alignment errors, or any combination thereof. The filter(s) 202, for instance, include a band-pass and/or low-pass filter with a cutoff frequency that is suitable for removing one or more types of high-frequency artifact.

In various cases, the input features 122 include the filtered sequence read data 114 and/or the filtered transformed data 118. In some examples, the input features 122 include one or more images representing the filtered sequence read data 114 and/or the filtered transformed data 118. According to some cases, the input features 122 include one or more features derived based on the filtered sequence read data 114 and/or the filtered transformed data 118. For example, the feature selector 120 may include a peak detector 204, a trough detector 206, a distance metric calculator 208, a genomic feature detector 210, or any combination thereof, configured to generate at least a portion of the input features 122 based on the filtered sequence read data 114 and/or the filtered transformed data 118. Unless contradicted by context, it should be understood that any mention of the sequence read data 114 and/or the transformed data 118 may referred to unfiltered and/or filtered versions of the sequence read data 114 and/or the transformed data 118.

The peak detector 204, in various cases, is configured to detect peaks 212 in the data represented by the sequence read data 114 and/or the transformed data 118. Various types of peak detection methods can be utilized by the peak detector 204. For example, the peak detector 204 may identify the peaks by detecting all datapoints in a dataset that exceed a threshold (e.g., 50% of a maximum value of the dataset) and/or are larger than their respective neighboring datapoints. According to some cases, the peaks 212 identified by the peak detector 204 are indicated in the input features 122. For instance, the input features 122 may include a genomic position or other characteristic of the peaks 212 identified by the peak detector 204.

The trough detector 206, in various examples, is configured to detect troughs 214 in the data represented by the sequence read data 114 and/or the transformed data 118. Various types of trough detection methods can be utilized by the trough detector 206. For instance, the trough detector 206 may identify continuous segments of the sequence read data 114 and/or the transformed data 118 that are lower than a particular threshold (e.g., 25% of a maximum value of the dataset). The troughs 214 may be indicated in the input features 122. For example, the input features 122 may include a genomic position, start position, end position, or other characteristic of the troughs 214 identified by the trough detector 206.

In various cases, the distance metric calculator 208 is configured to compare the sequence read data 114 and/or the transformed data 118 with pre-classified data 216. The pre-classified data 216 may represent nucleic acid molecules obtained from another individual (e.g., not the subject) with a known condition. For instance, the pre-classified data 216 may be based on a sample obtained from an individual with a known cancer diagnosis. According to various cases, the pre-classified data 216 is in the same dimension as the sequence read data 114 and/or the transformed data 118.

According to various implementations, the distance metric calculator 208 is configured to generate a distance metric representing a similarity between the sequence read data 114 and the pre-classified data 216 and/or between the transformed data 118 and the pre-classified data 216. In some cases, the distance metric is low (e.g., close to 0) when the datasets are dissimilar, and high (e.g., approaching 1) when the datasets are similar. Various types of distance metrics are calculated by the distance metric calculator 208, such as a chi-squared distance, a Jensen-Shannon divergence, a Jaccard index, a Sorensen-Dice coefficient, or any combination thereof. In some cases, the datasets are convolved or cross-correlated together, and an area under the curve (AUC) or maximum of the resultant dataset is utilized as a distance metric.

In various cases, the distance metric calculator 208 is configured to generate the distance metric based on images of the datasets (e.g., an image of the transformed data 118 and an image of the pre-classified data 216). In some examples, the distance metric calculator 208 is configured to perform one or more image recognition techniques to identify the similarity between the datasets based on the images. For example, an image of the pre-classified data 216 may be one of a set of eigenimages generated by performing principal component analysis (PCA) on multiple images depicting sequence read data and/or transformed data from a population of multiple individuals. The image of the dataset to be classified (e.g., the image of the transformed data 118) is compared to the set of eigenimages to generate a set of weights (e.g., vectors generated by projecting the image on the set of eigenimages). The weights, for instance, may be included in the input features 122. In some cases, a distance metric (e.g., a Hamming distance, a Euclidian distance, or the like) representing a similarity between the weights of the image to be classified and weights representing projections of the pre-classified data 216 on the eigenimages is included in the input features 122. In some cases, images of the sequence read data 114 and/or the transformed data 118 are included in the input features 122.

The genomic feature detector 210 is configured to determine one or more genomic features of the subject by analyzing the sequence read data 114 and/or the transformed data 118. For example, the genomic feature detector 210 may calculate at least one of a mutational profile of the sample, a mutational signature of the sample, an MMRD probability score, a copy number state, a fraction unstable score, or the presence of one or more pathogenic variants by analyzing the sequence read data 114 and/or the transformed data 118. One or more of the genomic features may be included in the input features 122.

In various implementations, the input features 122 are utilized to identify a condition of the subject. For example, the input features 122 are provided to a classifier configured to predict whether the subject has one or more conditions, or does not have the one or more conditions. In some cases, the classifier includes one or more ML models.

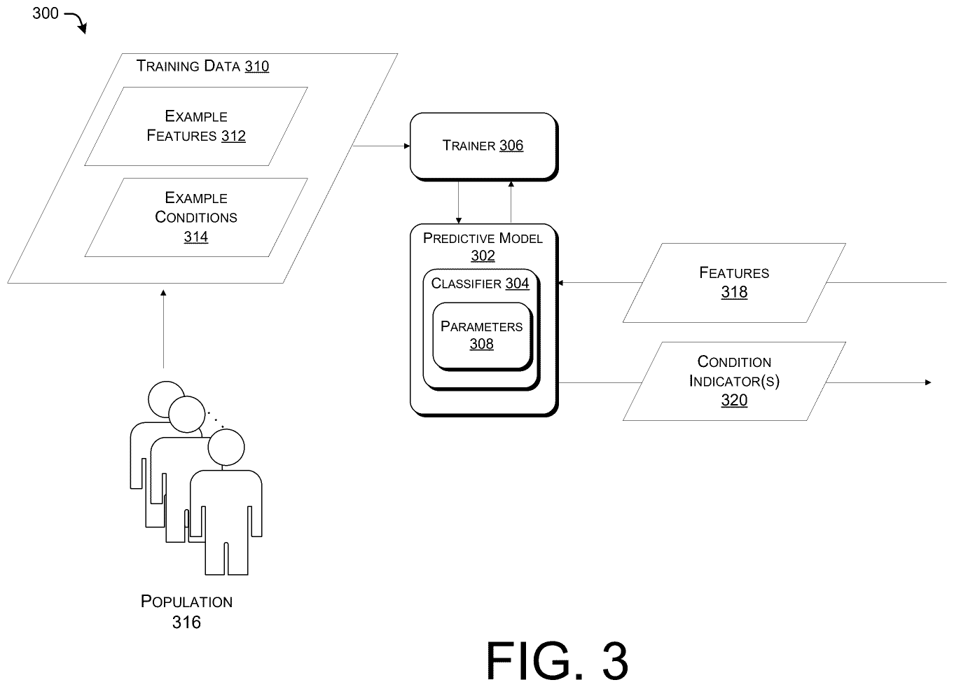

FIG. 3 illustrates an example environment 300 for training and utilizing a predictive model 302 to identify a condition of a subject. The predictive model 302, for instance, is the predictive model 124 described above with reference to FIG. 1. In various implementations, the predictive model 302 includes a classifier 304, which may include one or more ML models. A trainer 306, for instance, is configured to optimize various parameters 308 of the classifier 304 based on training data 310.

The training data 310 includes example features 312 and example conditions 314. The example features 312, in various cases, are obtained based on nucleic acid molecules of individuals within a population 316. In various examples, the example features 312 include, or are derived, based on transformations of sequence read data of the nucleic acid molecules into an alternate domain (e.g., transformations of the sequence read data from a spatial domain to a frequency or wavelet domain). The example conditions 314 may include indications of conditions of the individuals within the population 316. For example, the example conditions 314 may include indications of whether the individuals within the population 316 have one or more diseases. In some cases, the example conditions 314 may be generated based on clinical evaluations of the individuals within the population 316, such as by one or more care providers.

The classifier 304 include one or more model types. For instance, the classifier 304 include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 304 is defined according to the parameters 308. For example, the parameters 308 may include weights, thresholds, filters, kernels, or other data objects that are utilized to perform operations of the classifier 304.

In some implementations, the classifier 304 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 308 may include k (e.g., the number of neighbors compared to the new data point), the threshold distance, and so on.

In various cases, the classifier 304 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 308.

In some cases, the classifier 304 include a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 308, for example, include a threshold proximity within which a new data point is classified within a cluster, a density of points used to define a cluster, and the like.

In various examples, the classifier 304 include a principal component analysis model. In various implementations, a principal component analysis defines a collection principal components of unit vectors within a coordinate space based on a data set (e.g., training data). The model, for example, is an orthogonal linear transformation of the data set. Various weights of the model, for example, are included in the parameter(s) 308.

The classifier 304, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 308.

The classifier 304, for example, includes a random forest. The random forest, for instance, includes multiple decision trees that classify data in an ensemble fashion. In various implementations, the decision trees are defined by the parameter(s) 308.

In various implementations of the present disclosure, the trainer 306 is configured to optimize the parameters 308 based on the training data 310. For example, the trainer 306 may input first example features (corresponding to a first individual among the population 316) among the example features 312 into the predictive model 302, and may receive a predicted condition of the first individual as a result of computations performed using the predictive model 302. The trainer 306 may compute a loss (e.g., determine a discrepancy) between a first example condition (corresponding to the first individual) among the example conditions 314 and the predicted condition. Further, the trainer 306 may alter the parameters 308 in order to minimize the loss. In various cases, the trainer 306 optimizes the parameters 308 iteratively based on the entire set of the training data 310.

In various implementations, the optimization of the parameters 308 enables the predictive model 302 to identify predictive attributes of the example features 312 that are correlated to or otherwise associated with the example conditions 314. For instance, the predictive model 302 may determine that a particular peak pattern represented in transformed data among the example features 312 is highly correlated with adenosarcoma. The predictive model 302 may therefore classify conditions (e.g., cancers) based on features outside of the example features 312 by recognizing or otherwise identifying the predictive attributes.

Once the parameters 308 are optimized, the predictive model 302 may be ready to classify a new set of data. For example, the predictive model 302 may receive input data including features 318 of a subject. The features 318, for instance, may include one or more of the predictive attributes that are relevant for classifying a condition of the subject. According to various implementations, the features 318 are based on transforming sequence read data of the subject into the alternate domain. The predictive model 302 may perform various operations on the input data based on the trained classifier 304 and the optimized parameters 308. In various cases, the predictive model 302 outputs output data including one or more condition indicators 320 based on the features 318. The condition indicator(s) 320, for instance, include one or more predicted categories of a cancer experienced by the subject.

Although FIG. 3 is primarily described as referring to supervised learning, implementations are not so limited. In various cases, the training data 310 omits the example conditions 314 and the trainer 306 is configured to optimize the parameters 308 using the example features 312 and an unsupervised learning technique.

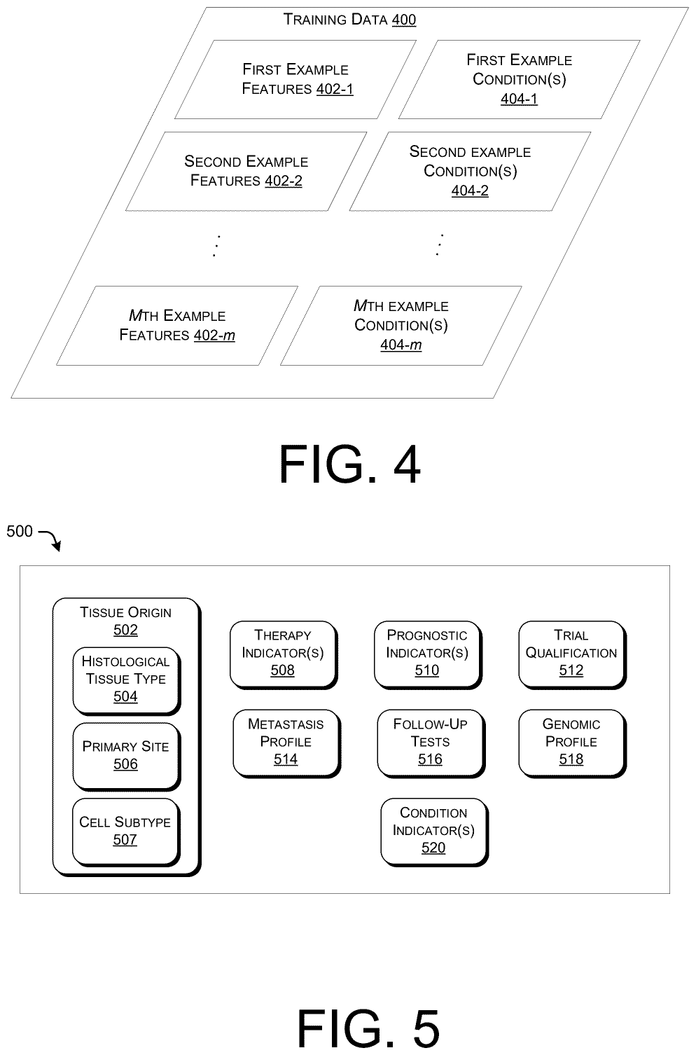

FIG. 4 illustrates an example of training data 400 utilized to train one or more ML models. For example, the training data 400 may be the training data 310 described above with reference to FIG. 3.

The training data 400, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 400 includes first to mth example features 402-1 to 402-m. For example, the first to mth example features 402-1 to 402-m include features derived from nucleic acid molecules in the respective m samples. In some cases, spatial domain data is obtained by sequencing the nucleic acid molecules. According to various implementations, the spatial domain data is converted to an alternate domain (e.g., a frequency or wavelet domain) to generate the first to mth example features 402-1 to 402-m.

The training data 400 may further include first to mth example conditions 404-1 to 404-m. The first to mth example conditions 404-1 to 404-m, for instance, include conditions of the individuals from which the m samples are obtained.

FIG. 5 illustrates an example report 500 summarizing predicted conditions of a subject. In various cases, the report 500 is the report 130 described above with reference to FIG. 1. The report 500, for instance, may be displayed to a patient and/or care provider. In some cases, the report 500 is generated based on features of a sample (e.g., a liquid biopsy sample) obtained from the subject.

In some cases, the subject is predicted to have a cancer. The report 500 includes a tissue origin 502 of the cancer. The tissue origin 502, for instance, indicates a histological tissue type 504, a primary site 506, cell subtype 507, or any combination, of the cancer.

In various cases, the report 500 includes one or more therapy indicators 508. For instance, the therapy indicator(s) 508 convey whether the cancer is predicted to be resistant to one or more predetermined therapies and/or whether the cancer is predicted to be responsive to one or more predetermined therapies.

In some examples, the report 500 includes one or more prognostic indicators 510. The prognostic indicator(s) 510, for instance, indicate a prognosis of the subject in view of the categorized cancer. For example, the prognostic indicator(s) 510 may indicate a survivability, a recoverability, a quality of life indicator, or other information indicative of the prognosis of the subject.

The report 500 may include a trial qualification 512 of the subject. The trial qualification 512, for instance, indicates whether the subject is predicted to qualify for a predetermined clinical trial.

The report 500, in various implementations, includes a metastasis profile 514 of the subject. The metastasis profile 514, for instance, indicates a likelihood that the cancer will metastasize (e.g., at a particular point in time), one or more tissues in which the cancer is predicted to metastasize, or the like.

In various cases, the report 500 includes recommended follow-up tests 516. For example, the report 500 may include a recommendation to perform whole genome sequencing on the subject, particularly in cases if the cancer cannot be categorized above a threshold certainty.

The report 500 may include a genomic profile 518 of the subject. In various cases, the genomic profile 518 includes or is generated based on the results of non-fragmentomic analyses of the subject.

In various implementations, the report 500 includes at least one condition indicator 520. The condition indicator(s) 520, for instance, indicate one or more predicted conditions of the subject. For instance, if the subject is predicted to have a type of cancer, the condition indicator(s) 520 may indicate the type of cancer. Other types of conditions may also be noted in the condition indicator(s) 520, such as a general health of the subject, a genomic age of the subject, a risk that the subject will develop a disease, a predicted pathology of the subject, a predicted pathology subtype of the subject, a predicted survivability of the subject, a predicted effective therapy to treat the predicted pathology of the subject, a predicted stage of the predicted pathology of the subject, a predicted grade of the predicted pathology of the subject, an ECOG performance status of the subject. Various types of pathological conditions may be indicated in the condition indicator(s) 520, such as a cancer, a genetic disorder, diabetes, hypertension, heart disease, a respiratory disease, an infectious disease, or an autoimmune disease.

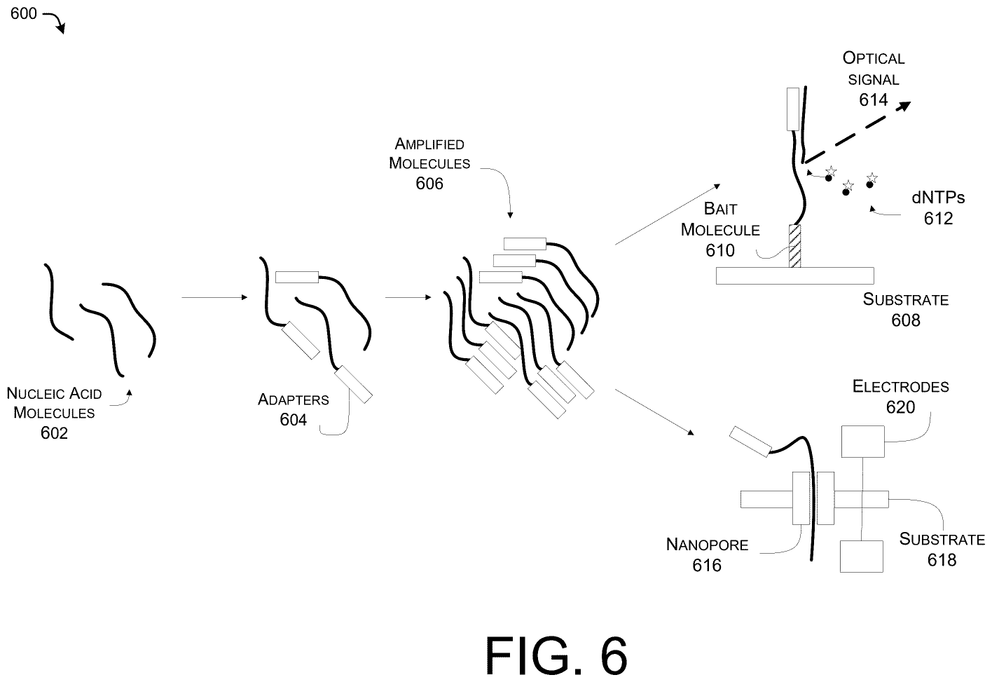

FIG. 6 illustrates an example environment 600 for sequencing various nucleic acid molecules 602. In various implementations, the nucleic acid molecules 602 include cfDNA and/or gDNA. For instance, the nucleic acid molecules 602 may include ctDNA. The nucleic acid molecules 602, in various cases, are extracted from a sample, such as a biological sample obtained from a subject. In some implementations, the nucleic acid molecules 602 include DNA that is complementary to RNA present in the sample.

The nucleic acid molecules 602, in various cases, are ligated with adapters 604. For examples, the adapters 604 are hybridized to the nucleic acid molecules 602. The adapters 604, for example, include additional nucleic acid molecules. In various implementations, the adapters 604 have a shorter length than the nucleic acid molecules 602 being sequenced. For instance, the adapters 604 include amplification primers, flow cell adapter sequences, substrate adapter sequences, or sample index sequences. Although FIG. 6 illustrates adapters 604 being ligated to one end of each of the nucleic acid molecules 602, implementations are not so limited. For example, the adapters 604 may be ligated to both ends of each of the nucleic acid molecules 602.

In various examples, the nucleic acid molecules 602 ligated with the adapters 604 are amplified in order to generate amplified molecules 606. Various amplification techniques can be performed. For instance, the amplified molecules 606 are generated using PCR, a non-PCR amplification technique, an isothermal amplification technique, or any combination thereof.

Amplified molecules 606 may be captured by bait molecules 610 and sequenced. In some implementations, the amplified molecules 606 are sequenced via sequencing-by-synthesis. In various cases, fluorescently tagged deoxyribonucleotide triphosphates (dNTP) 612 are utilized to synthesize a strand that is complementary to DNA strands bound to the substrate 608. When a dNTP 612 is added to the strand (e.g., by an enzyme), the dNTP 612 emits an optical signal 614. In various implementations, the frequency of the optical signal 614 is dependent on the type of dNTP 612 from which the optical signal 614 is emitted. By detecting the optical signals 614 as the strand is being synthesized, the sequence of the original nucleic acid molecules 602 can be derived.

In some implementations, the amplified molecules 606 are sequenced via nanopore sequencing. For instance, the amplified molecules 606 are directed through a nanopore 616 extending through a substrate 618. In various cases, the amplified molecules 606 are negatively charged, such that they can be directed through the nanopore 616 by imposing an electrical field across the substrate 618. In various cases, the amplified molecules 606 and the nanopore 616 are in the presence of a charged solution. Thus, charged solutes traveling through the nanopore 616 can be monitored by reviewing an electrical signal (e.g., a current) sensed between electrodes 620 on either side of the substrate 618. As an amplified molecule 606 is directed through the nanopore 616, the individual bases within the amplified molecule 606 will block the nanopore 616, which may decrease the amount of charged solutes traveling through the nanopore 616 and consequently, the magnitude of the electrical signal detected by the electrodes 620. Each of the four types of bases within the amplified molecules 606, may block the nanopore 616 to a different extent. Therefore, the sequences of the nucleic acid molecules 602 can be derived by analyzing the measured electrical signal with respect to time as the amplified molecules 606 are directed through the nanopore 616.

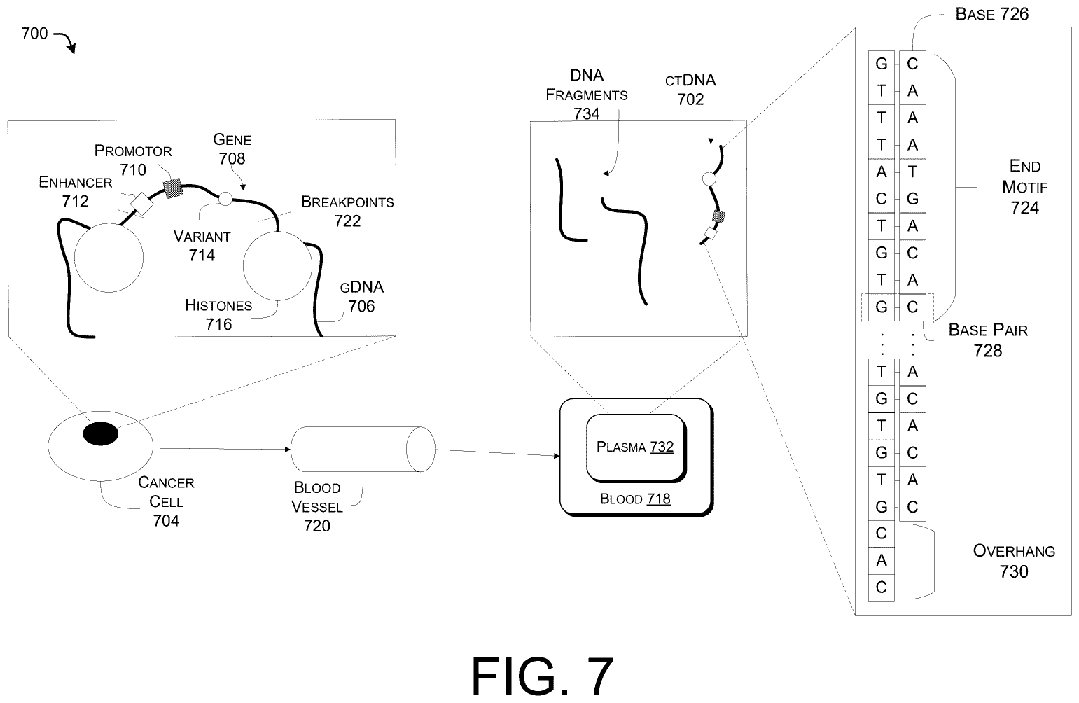

FIG. 7 illustrates an example environment 700 illustrating ctDNA 702, which can be utilized to a condition of a subject. For instance, the ctDNA 702 may be included in the nucleic acid molecules 110 described above with reference to FIG. 1.

In various implementations, a cancer cell 704 within the subject includes genomic DNA (gDNA) that is expressed by the cancer cell 704. For example, the gDNA 706 may include various sequences, such as a gene 708, a promoter 710, an enhancer 712, and a variant 714. For example, the variant 714 is part of the gene 708. In addition, various epigenetic factors impact expression of the gene 708 as well as other genes within the gDNA 706. For example, the gDNA 706 may be packaged within the nucleus of the cancer cell 704 with various histones 716. When the gene 708 is expressed, a portion of the gDNA 706 including the gene 708, the promotor 710, the enhancer 712, and the variant 714 may be exposed to proteins within the nucleus, such as RNA transcriptase. In various cases, the portion of the gDNA 706 is unwrapped or otherwise unpackaged from the histones 716. Thus, the expression of the gene 708 (e.g., the amount of mRNA generated by RNA transcriptase based on the gene 708 within the cancer cell 704) is linked to the frequency or time at which the portion of the gDNA 706 is exposed.

The cancer cell 704, for example, may die. The contents of the cancer cell 704, including the gDNA 706, may be released. In various cases, the gDNA 706 is released into blood 718 that flows through a blood vessel 720 of the subject. When the gDNA 706 is released from the nucleus of the cancer cell 704, the gDNA 706 is degraded due to various biophysical and/or biochemical factors. For example, the blood 718 may include various enzymes that cut the gDNA 706 into the ctDNA 702. In various cases, other mechanical, chemical, or thermal conditions in the blood 718 divide the gDNA 706 into the ctDNA 702. For example, these conditions divide the gDNA 706 into fragments at various breakpoints 722.

Notably, the presence and location of the histones 716 may impact the sequences of the ctDNA 702 that are observed in the blood 718. The breakpoints 722, for example, are more likely to occur at edges of a sequence of the gDNA 706 that is exposed by the histones 716. Therefore, the sequence of the ctDNA 702 is indicative of the expression of mRNA and other functional RNA in the cancer cell 704. By reviewing the ctDNA 702, the expression of the cancer cell 704 can be determined without performing RNA sequencing, in some cases. In various examples, the expression of the cancer cell 704 is relevant to the condition of the subject.

In addition, the sequences at or near the breakpoints 722 are indicative of expression of the cancer cell 704. For example, the ctDNA 702 may include an end motif 724. The end motif 724 may be defined as a sequence of bases 726 and/or base pairs 728 that extend from an end of the ctDNA 702. The end motif 724, for example, has a predetermined length that is in a range of 1 to 30 bases and/or base pairs. In various implementations, the ctDNA 702 is a double-stranded DNA molecule with an overhang 730. The overhang 730, for instance, includes one or more bases 726 of one ssDNA molecule that extends beyond the corresponding end of the other ssDNA molecule. In some cases, the end motif 724 is defined as the sequence of bases in a single ssDNA within the ctDNA 702 or a sequence of complementary base pairs in both ssDNA within the ctDNA 702. As described herein, the term “endpoint” may refer to at least one of the bases 726 in the end motif 724 and/or overhang 730 of a DNA fragment in a sample.

In various implementations, the ctDNA 702 is obtained from a sample of plasma 732 in the blood 718 of the subject. The plasma 732, for example, includes various DNA fragments 734 including the ctDNA 702. In some cases, the DNA fragments 734 include various cfDNA, such as cfDNA released from non-cancerous cells.

By sequencing the ctDNA 702, various fragmentomic features may be obtained. These fragmentomic features can be utilized to categorize the cancer cell 704, thereby identifying a condition of the subject from which the cancer cell 704 was present. In various cases, the fragmentomic features include the presence of at least a portion of the gene 708 in the ctDNA 702. In some cases, the fragmentomic features include the presence of at least a portion of the promotor 710, the enhancer 712, or the variant 714 in the ctDNA 702. In some cases, the fragmentomic features include the presence or sequence of the end motif 724. Other fragmentomic features are described elsewhere herein.

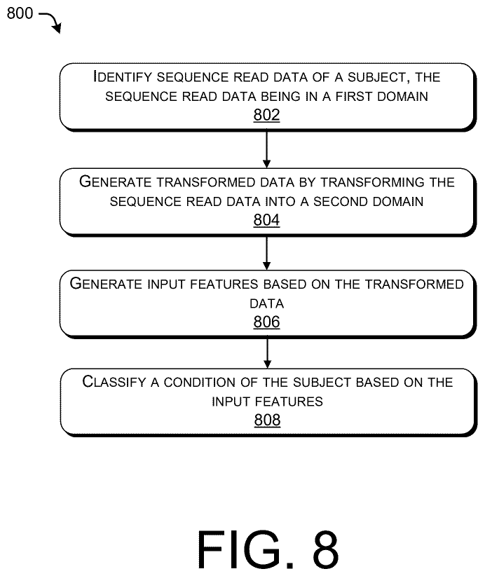

FIG. 8 illustrates an example process 800 for identifying a condition of a subject using transformed data. In various implementations, the process 800 is performed by an entity including at least one processor, at least one computing device, a medical device, the sequencer 112, the data transformer 116, the feature selector 120, the predictive model 124, the report generator 128, the clinical device 132, the predictive model 302, or any combination thereof.

At 802, the entity identifies sequence read data of a subject. The sequence read data, for instance, is in a first domain. In some cases, the entity generates the sequence read data. For instance, the entity receives a plurality of nucleic acid molecules in a sample from a subject. The sample may include a liquid biopsy sample, a tissue sample, or a combination thereof. The nucleic acid molecules, for instance, include DNA fragments in a liquid biopsy sample. One or more adapters are ligated onto at least some of the nucleic acid molecules. The ligated molecules are amplified and captured. In various cases, all or a subset of the captured molecules are sequenced to obtain a plurality of sequence reads that represent the sequenced amplified nucleic acid molecules, thereby generating the sequence read data. In particular examples, the sequence read data includes endpoint counts of fragments at multiple genomic positions within at least one locus of the genome of the sample.

At 804, the entity generates transformed data by transforming the sequence read data into a second domain. The second domain, for instance, is an alternate domain that is different than the first domain. In some cases, the entity performs a Fourier transform (e.g., an FFT, an STFT, a DFT, etc), a Hartley transform, a Laplace transform, a Mellin transform, a wavelet transform, or any combination thereof on the sequence read data.

In some examples, the entity filters the transformed data. In some cases, the entity may multiple, convolve, cross-correlate, or any combination thereof, a filter with the transformed data.

At 806, the entity generates input features based on the transformed data. In some cases, the entity converts the transformed data into an image (e.g., a spectrogram or other image representing the data) and generates the input features using a CNN configured to detect the input features. In some cases, the entity identifies one or more peaks and/or one or more troughs of the transformed data, and indicates the peak(s) and/or trough(s) in the input features. In some cases, the entity identifies at least one distance metric by comparing the transformed data and at least one set of pre-classified data. For example, the at least one set of pre-classified data includes a dataset obtained from an individual that has a particular condition (e.g., a particular type or subtype of cancer) as well as a dataset obtained from an individual that does not have the particular condition. The distance metric(s), for instance, are included in the input features.

At 808, the entity classifies a condition of the subject based on the input features. In some cases, the entity utilized an ML-based classifier to predict whether the subject has the condition. The ML-based classifier, for instance, is pre-trained based on data obtained from a population of individuals that omits the subject. In some cases, the classifier includes at least one of an ANN, a logistic regression model, a decision tree, a KNN model, a support vector machine (SVM), or a naïve Bayes classifier. In some cases, the classifier outputs a likelihood that the subject has a particular condition (or the absence of a particular condition). In some cases, the classifier outputs an indication that the subject has the particular condition (or its absence) when the likelihood exceeds a threshold likelihood.

Various types of conditions can be predicted using the process 800. In some cases, the entity predicts whether the subject has a pathological condition, such as a cancer, a genetic disorder, diabetes, hypertension, heart disease, a respiratory disease, an infectious disease, an autoimmune disease, or any combination thereof. In some cases, the entity predicts a subtype, metastasis profile, survivability, symptom, risk of developing, stage, grade, or ECOG performance of the predicted pathological condition. In some cases, the entity predicts whether a particular therapy will be effective to treat the pathological condition and/or whether the pathological condition is resistant to the particular therapy. In some cases, the entity predicts a general health, genomic age, or risk of developing a pathological condition, of the subject. According to some cases, the entity generates a comprehensive metric or “score” indicating a level of health or disease of the subject.

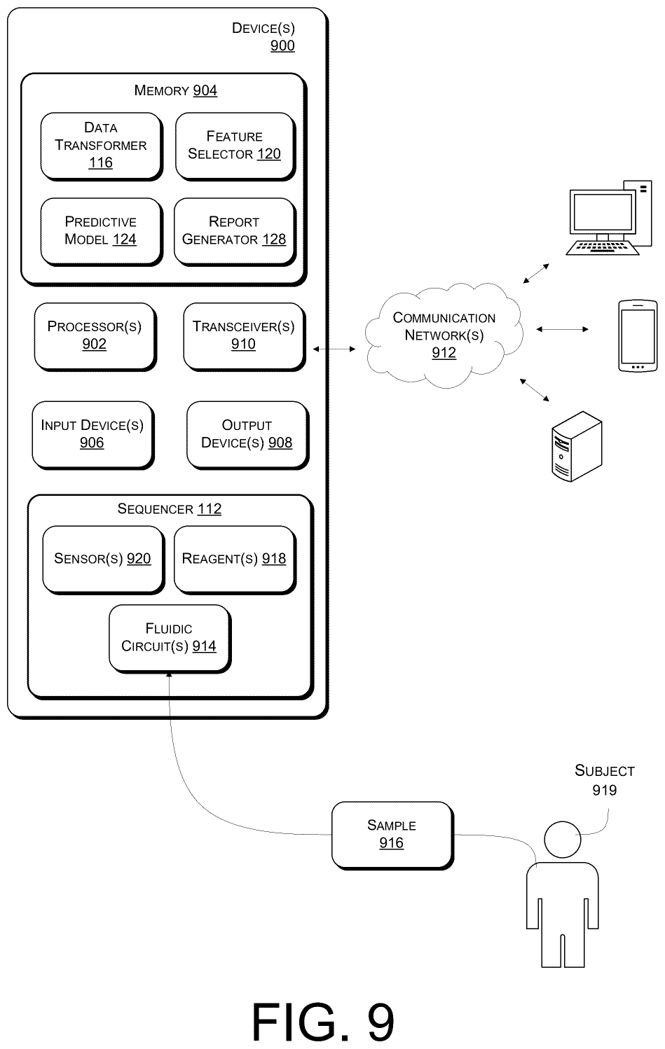

FIG. 9 illustrates one or more devices 900 configured to perform various operations described herein. The device(s) 900 include one or more processor(s) 902. In some implementations, the processor(s) 902 includes a central processing unit (CPU), a graphics processing unit (GPU), both CPU and GPU, or other processing unit or component known in the art.

The processor(s) 902 is operably connected to memory 904. In various implementations, the memory 904 is volatile (such as random access memory (RAM)), non-volatile (such as read only memory (ROM), flash memory, etc.) or some combination of the two. The memory 904 stores instructions that, when executed by the processor(s) 902, causes the processor(s) 902 to perform various operations. In various examples, the memory 904 stores methods, threads, processes, applications, objects, modules, any other sort of executable instruction, or a combination thereof. In some cases, the memory 904 stores files, databases, or a combination thereof. In some examples, the memory 904 includes, but is not limited to, RAM, ROM, electrically erasable programmable read-only memory (EEPROM), flash memory, or any other memory technology. In some examples, the memory 904 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 902. For instance, the memory 904 stores instructions that, when executed by the processor(s) 902, causes the processor(s) 902 to perform operations of the data transformer 116, the feature selector 120, the predictive model 124, the report generator 128, or any combination thereof.

The processor(s) 902 is operably connected to one or more input devices 906 and one or more output devices 908. Collectively, the input device(s) 906 and the output device(s) 908 function as an interface between at least one user and the device(s) 900. The input device(s) 906 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 908 includes at least one of a display, a speaker, a haptic output device, a printer, or any combination thereof. In various examples, the processor(s) 902 causes a display among the input device(s) 906 to visually output various data described herein. In some implementations, the input device(s) 906 includes one or more touch sensors, the output device(s) 908 includes a display screen, and the touch sensor(s) are integrated with the display screen.

In various implementations, the processor(s) 902 is operably connected to one or more transceivers 910 that transmit and/or receive data over one or more communication networks 912. For example, the transceiver(s) 910 includes a network interface card (NIC), a network adapter, a local area network (LAN) adapter, or a physical, virtual, or logical address to connect to the various external devices and/or systems. In various examples, the transceiver(s) 910 includes any sort of wireless transceivers capable of engaging in wireless communication (e.g., radio frequency (RF) communication). For example, the communication network(s) 912 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 910 includes other wireless modems, such as a modem for engaging in WI-FI®, WIGIG®, WIMAX®, BLUETOOTH®, or infrared communication over the communication network(s) 912.

The device(s) 900 may further include the sequencer 112. In various implementations, the sequencer 112 includes one or more fluidic circuits 914 configured to receive a sample 916 derived from a subject 919. The sequencer 112, in various cases, may be configured to generate data indicative of one or more sequences of nucleic acid molecules (e.g., DNA and/or RNA) present in the sample 916. In various cases, the sequencer 112 introduces one or more reagents 918 to the fluidic circuit(s) 914 in order to prepare for and perform sequencing of the nucleic acid molecules. Further, the sequencer 112 may include one or more sensors 920 configured to measure or otherwise detect detection signals from the fluidic circuit(s) 914, which may be indicative of the sequences of the nucleic acid molecules. According to various implementations, the sensor(s) 920 may further include one or more ADCs. The sequencer 112, in various cases, outputs sequence read data to the processor(s) 902 for additional processing.

### Experimental Example—Predicting Prostate Cancer Status Using Transformed Fragmentomics Data

Various analyses on DNA fragments in a sample can be used to infer DNA expression of cells in the sample. Nucleosome density is indicative of the openness of nuclear DNA for transcription in a cell. When a cell lyses, portions of the nuclear DNA exposed by nucleosomes may be preferentially generated into DNA fragments. Therefore, the sequence and/or length of DNA fragments in a sample may be analyzed to infer cell expression patterns.

Various metrics can be derived based on DNA fragments. Window protection score (WPS), for instance, is a known measure of relative read endpoint density. For a given window size in a genomic locus, WPS represents the relative density of fragments spanning the window to those that do not. Relative read depth is also associated with accessibility of a genomic region (e.g., a promoter) or occupancy of a binding site. Fragment endpoint count may also be relevant to DNA expression.

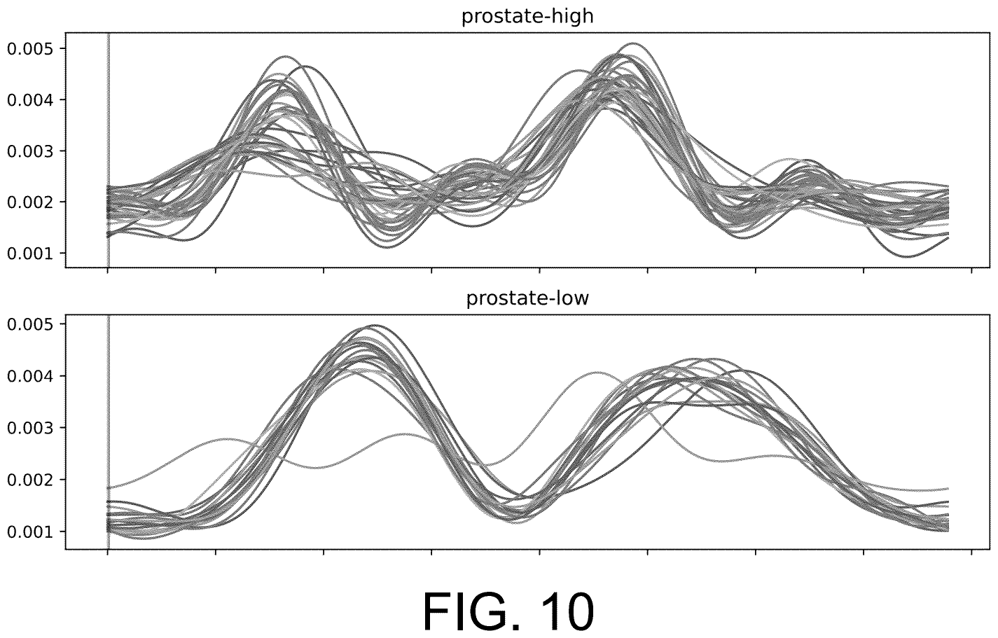

This Example describes a method of detecting periodic deviations in sequencing data by time-frequency transform. As described herein, these deviations may be indicative of prostate cancer status. FIG. 10 illustrates an example of endpoint data for nucleic acid samples in first liquid biopsy samples (upper) and endpoint data for nucleic acid samples in second liquid biopsy samples (lower), wherein the first liquid biopsy samples have relatively high percentages of prostate cancer tumor DNA and the second liquid biopsy samples have relatively low percentages of prostate cancer tumor DNA. Thirty “high shed” (the first liquid biopsy samples) and seventeen “low shed” (the second liquid biopsy samples) are represented in FIG. 10. Notably, raw endpoint data includes high-frequency noise, which can be removed using a low-pass filter.

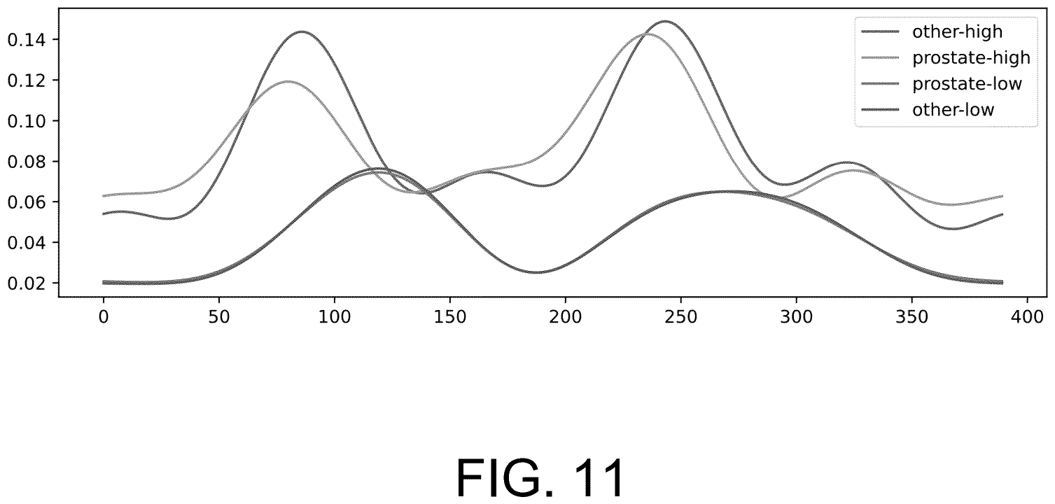

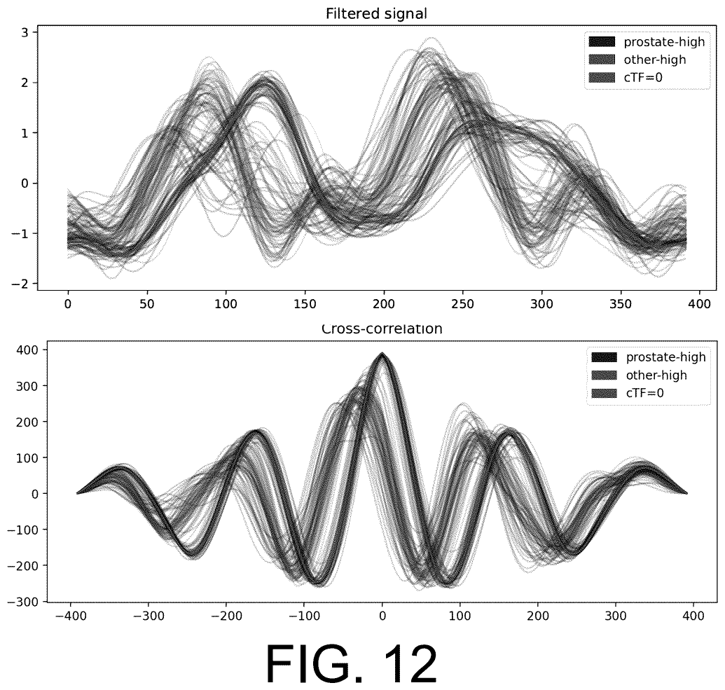

FIG. 11 illustrates shifts in periodicity between the liquid biopsy samples of FIG. 10. Specifically, FIG. 11 illustrates the results from the liquid biopsy samples of FIG. 10 averaged and plotted together, demonstrating a shift to the left for high shed prostate samples. Other tumor types are also shown. In some regions, shifts in periodicity appear to be correlated to the prevalence of tumor DNA. For instance, the peaks of the high shed samples are shifted to the left of the peaks of the low shed samples.

FIG. 12 compares raw signal to cross-correlations of fragmentomics data (e.g., endpoint counts) for high shed prostate cancer samples, other non-prostate cancer high shed samples, and samples without circulating tumor fragments. As shown, high shed biopsies show consistent differences in comparison with low shed biopsy samples. Thus, cross-correlation of these samples may be indicative of the cancer status of the subjects from which the samples are obtained.

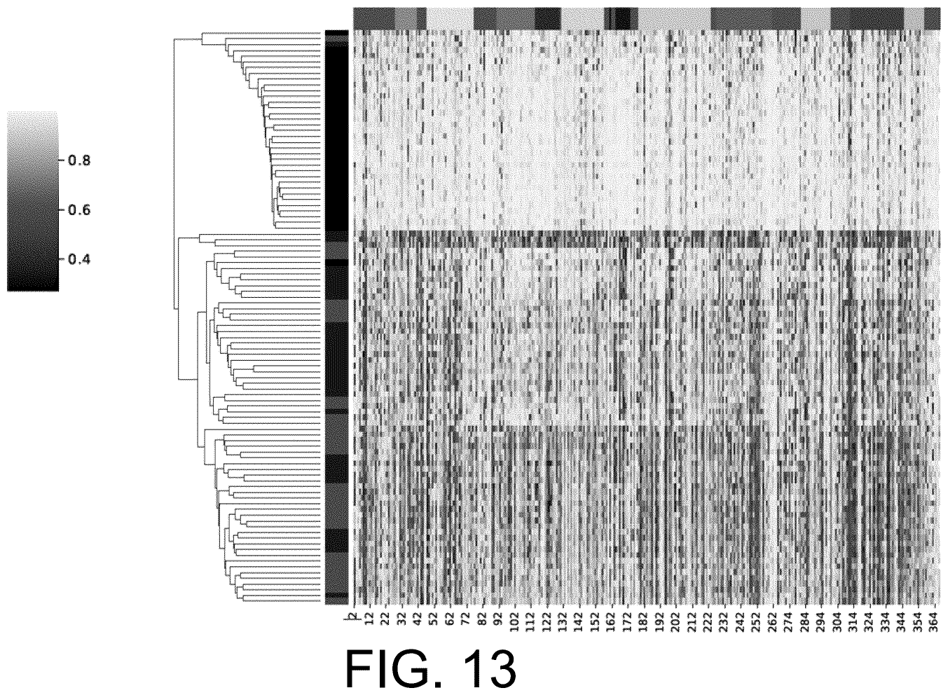

FIG. 13 illustrates the nearly 400 regions of the X chromosome that were analyzed in the Example for features that are predictive of prostate cancer status.

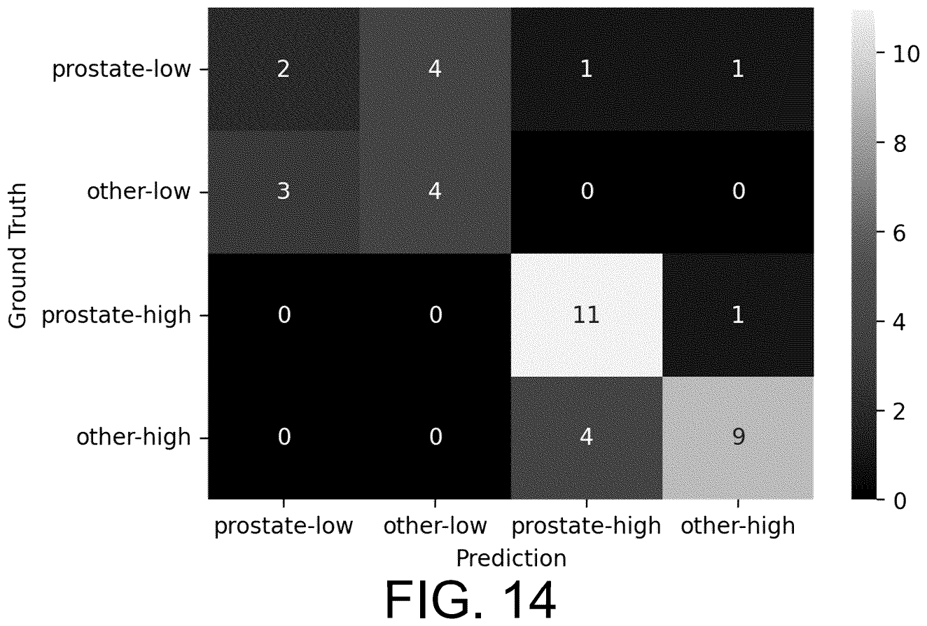

FIG. 14 illustrates results of logistic regression, showing these features result in reasonable predictive performance for assessing prostate cancer status.

### Example Clauses

The following clauses provide various non-limiting implementations of the present disclosure:

1. A method, including: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data, the sequence read data being in a spatial domain corresponding to genomic position within at least one locus of a genome of the sample; receiving, at one or more processors, the sequence read data for the plurality of sequence reads; generating, by the one or more processors, transformed data by performing a Fast Fourier Transform (FFT) on the sequence read data, the transformed data representing the sequence read data in a frequency domain; generating, by the one or more processors, input features based on the transformed data; and determining, using a classifier executed by the one or more processors, a condition of the subject based on the input features.
2. The method of clause 1, wherein the plurality of nucleic acid molecules include DNA fragments obtained from a liquid biopsy sample obtained from the subject, and wherein the sequence read data includes endpoint counts of the DNA fragments at multiple genomic positions within the at least one locus of the genome of the sample.
3. The method of clause 1 or 2, wherein generating, by the one or more processors, the input features based on the transformed data includes at least one of: inputting, into a convolutional neural network (CNN) configured to detect the input features, an image representing the transformed data; identifying one or more peaks of the transformed data; identifying one or more troughs of the transformed data; determining a first distance metric by comparing the transformed data and first pre-classified data, the first pre-classified data being in the frequency domain and being transformed from sequence read data of a first individual having the condition; or determining a second distance metric by comparing the transformed data and second pre-classified data, the second pre-classified data being in the frequency domain and transformed from sequence read data of a second individual that does not have the condition.
4. The method of clause 3, wherein determining the first distance metric includes: generating first alignment data by cross-correlating and/or convolving the transformed data and the first pre-classified data; and determining at least one of a peak of the first alignment data or a mean of the first alignment data, and wherein determining the second distance metric includes: generating second alignment data by cross-correlating and/or convolving the transformed data and the second pre-classified data; and determining at least one of a peak of the second alignment data or a mean of the second alignment data.
5. The method of any of claims 1 to 4, wherein the condition includes a cancer type or subtype of the subject.
6. The method of any of claims 1 to 5, further including at least one of: removing a high-frequency artifact from the transformed data by multiplying a low-pass filter with the transformed data, or removing a low-frequency artifact from the transformed data by multiplying a high-pass filter with the transformed data.
7. A method, including: identifying sequence read data of a sample obtained from a subject, the sequence read data being in a spatial domain corresponding to genomic position; generating transformed data by transforming the sequence read data into an alternative domain; generating input features based on the transformed data; and classifying, using a classifier, a condition of the subject based on the input features.
8. The method of clause 7, wherein the sequence read data corresponds to a single genomic locus.
9. The method of clause 7 or 8, wherein the sequence read data includes endpoint counts of fragments in the sample at multiple genomic positions.
10. The method of clause 9, wherein the endpoint counts include left endpoint counts and/or right endpoint counts.
11. The method of any of clauses 7 to 10, wherein the sequence read data includes read depth of fragments in the sample at multiple genomic positions.
12. The method of any of clauses 7 to 11, wherein the sequence read data indicates lengths of fragments in the sample.
13. The method of any of clauses 7 to 12, wherein the sequence read data indicates a quantity and/or presence of variants present in fragments in the sample at multiple genomic positions.
14. The method of clause 13, wherein the variants include at least one of a substitution, an insertion, a deletion, a copy number alternation, or a rearrangement of fusion.
15. The method of any of clauses 7 to 14, further including: receiving a plurality of nucleic acid molecules obtained from the sample; ligating one or more adapters onto one or more nucleic acid molecules from the plurality of nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating the sequence read data for a genome of the sample.
16. The method of clause 15, wherein the one or more adapters include amplification primers, flow cell adapter sequences, substrate adapter sequences, or sample index sequences.
17. The method of clause 15 or 16, wherein the captured nucleic acid molecules are captured from the amplified nucleic acid molecules by hybridization to one or more bait molecules.
18. The method of clause 17, wherein the one or more bait molecules include one or more additional nucleic acid molecules, each of the one or more additional nucleic acid molecules including a region that is complementary to a region of a captured nucleic acid molecule.
19. The method of any of clauses 15 to 18, wherein amplifying the one or more ligated nucleic acid molecules includes performing a polymerase chain reaction (PCR) amplification technique, a non-PCR amplification technique, or an isothermal amplification technique.
20. The method of any of clauses 15 to 19, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
21. The method of any of clauses 15 to 20, wherein sequencing the captured nucleic acid molecules includes next generation sequencing (NGS).
22. The method of any of clauses 15 to 21, wherein the sequencer includes a next generation sequencer.
23. The method of any of clauses 15 to 22, wherein sequencing the captured nucleic acid molecules includes sequencing-by-synthesis or nanopore sequencing.
24. The method of any of clauses 7 to 23, further including: generating ligated molecules by ligating adapters onto nucleic acid molecules of the sample; generating amplified ligated molecules by amplifying the ligated molecules; generating, using the amplified ligated molecules, detection signals; detecting, by at least one sensor, the detection signals; and generating the sequence read data based on the detection signals.
25. The method of clause 24, wherein the detection signals include electrical signals and/or optical signals.
26. The method of clause 24 or 25, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the sample.
27. The method of any of clauses 24 to 26, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the sample.
28. The method of any of clauses 24 to 27, wherein the sequence read data indicates a full genome or RNA transcriptome of the sample.
29. The method of any of clauses 24 to 28, wherein the sequence read data indicates a whole exome of the sample.
30. The method of any of clauses 24 to 29, wherein the sequence read data indicates a predetermined panel of genes of the sample.
31. The method of clause 30, wherein the predetermined panel includes one or more of TP53, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1.
32. The method of clause 30 or 31, wherein the predetermined panel includes one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRF11, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF. BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP. PD-1, PDGFR, PDGFRα, PDGFRβ, PD-L1, PI3Kδ, PIGF. PTCH, RAF, RANKL, RET, ROS1, SLAMF7, VEGF, VEGFA, or VEGFB.
33. The method of any of clauses 24 to 32, further including: receiving the sample.
34. The method of clause 33, wherein the sample includes a tissue biopsy sample, a liquid biopsy sample, or a normal control.
35. The method of clause 33 or 34, wherein the sample is a liquid biopsy sample and includes blood, plasma, cerebrospinal fluid, sputum, stool, urine, lymphatic fluid, or saliva.
36. The method of any of clauses 33 to 35, wherein the sample is a liquid biopsy sample and includes circulating tumor cells (CTCs).
37. The method of any of clauses 33 to 36, wherein the sample is a liquid biopsy sample and includes cell-free DNA (cfDNA), circulating tumor DNA (ctDNA), or any combination thereof.
38. The method of any of clauses 33 to 37, further including extracting DNA or RNA from the sample.
39. The method of clause 38, wherein the DNA includes genomic DNA or cDNA.
40. The method of clause 39, wherein the RNA includes messenger RNA, microRNA, or non-coding RNA.
41. The method of any of clauses 7 to 40, wherein generating the input features further includes: determining, based on the sequence read data, a mutational profile of the sample; inputting the mutational profile into a model, wherein the model is trained using training data related to a plurality of mutational signatures; and predicting one or more mutational signatures of the plurality of mutational signatures associated with the sample based on an output of the model, wherein the output of the model is associated with a dimensionality value that is less than a number of the plurality of mutational signatures, and wherein the features include the one or more mutational signatures.
42. The method of clause 41, wherein the model includes an autoencoder model.
43. The method of any of clauses 7 to 42, wherein generating the input features further includes: determining, based on the sequence read data, a mismatch repair deficiency (MMRD) probability score, the MMRD probability score being indicative of a functional deficiency in at least one mismatch repair gene, wherein the features include the MMRD probability score.
44. The method of clause 43, the input features being first input features, wherein determining, based on the sequence read data, the MMRD probability score includes: generating, by extracting two or more second features of the sequence read data, second input features; and inputting the second input features into a predictive model configured to generate the MMRD probability score based on the second input features.
45. The method of any of clauses 7 to 44, wherein generating the input features further includes: determining, based on the sequence read data, a copy number state, and wherein the input features further include the copy number state.
46. The method of clause 45, wherein determining, based on the sequence read data, the copy number state includes: generating, based on the sequence read data, a major allele coverage ratio and a minor allele coverage ratio; segmenting one or more nucleic acid sequences associated with the sequence read data into segments; generating copy number grid model input features including: a sum of the major allele coverage ratio and the minor allele coverage ratio; and a difference of the major allele coverage ratio and the minor allele coverage ratio; fitting copy number grid models including allowed copy number states to the copy number grid model input features; selecting a copy number grid model among the copy number grid models; and assigning the copy number state for at least a portion of the one or more nucleic acid sequences based on the selected copy number grid model.
47. The method of any of clauses 7 to 46, wherein generating the input features further includes determining, based on the sequence read data, a fraction unstable score.
48. The method of clause 47, wherein determining the fraction unstable score further includes determining an MSI fraction.
49. The method of any of clauses 7 to 48, wherein generating the input features further includes: identifying, by analyzing the sequence read data, one or more pathogenic variants in at least one of POLE, TP53, CTNNNB1, L1CAM, PTEN, ERBB2, PMS2, MSH2, MSH6, MLH1, an estrogen receptor (ER) gene, or a progesterone receptor (PR) gene.
50. The method of any of clauses 7 to 49, wherein the sample is obtained from a tumor of the subject.
51. The method of any of clauses 7 to 50, wherein generating the transformed data by transforming the sequence read data into the alternative domain includes: generating the transformed data by performing a Fourier transform on the sequence read data, the alternative domain being a frequency domain.
52. The method of clause 51, wherein performing the Fourier transform on the sequence read data includes performing a Fast Fourier Transform (FFT) on the sequence read data.
53. The method of any of clauses 7 to 52, wherein the input features are generated based at least in part on pre-classified data, the pre-classified data being generated by: identifying training sequence read data associated with samples corresponding to a plurality of individuals omitting the subject; generating training transformed data by transforming the training sequence read data into the alternative domain; and generating the pre-classified data by labeling the training transformed data with labels indicative of conditions of the plurality of subjects.
54. The method of clause 53, further including: training a machine learning (ML) model to identify attributes, indicated by the training transformed data, that are predictive of the conditions of the plurality of subjects, wherein the input features are instances of the attributes identified via the training of the ML model.
55. The method of any of claims 7 to 54, wherein generating the transformed data by transforming the sequence read data into the alternative domain includes: performing at least one of a Fourier transform, a short-time Fourier transform (STFT), a discrete Fourier transform (DFT), a Hartley transform, Laplace transform, a Mellin transform, or a Wavelet transform on the sequence read data.
56. The method of any of claims 7 to 55, wherein generating the input features based on the transformed data includes: determining a distance metric by comparing the transformed data and pre-classified data, the pre-classified data being in the alternate domain.
57. The method of clause 56, wherein determining the distance metric by comparing the transformed data and the pre-classified data includes: generating alignment data by cross-correlating and/or convolving the transformed data and the pre-classified data; and determining the distance metric based on the alignment data.
58. The method of clause 57, wherein determining the distance metric based on the alignment data includes determining at least one peak of the alignment data and/or at least one mean of the alignment data.
59. The method of clause 57 or 58, wherein determining the distance metric by comparing the transformed data and the pre-classified data includes determining a similarity between the transformed data and the pre-classified data.
60. The method of any of clauses 57 to 59, wherein the pre-classified data is based on a sample obtained from an individual that has the condition and/or has a predetermined subtype of the condition.
61. The method for any of clauses 57 to 60, wherein the pre-classified data is based on a sample obtained from an individual that lacks the condition.
62. The method of any of clauses 7 to 61, wherein generating the input features based on the transformed data includes: identifying at least one peak of the transformed data.
63. The method of any of clauses 7 to 62, wherein generating the input features based on the transformed data includes: generating a digital image based on the transformed data.
64. The method of clause 63, wherein generating the input features based on the transformed data further includes: extracting the input features from the digital image by inputting the digital image into a convolutional neural network (CNN).
65. The method of clause 64, wherein the CNN includes multiple layers, an individual layer among the multiple layers including a kernel defined by one or more parameters, and wherein extracting the input features from the digital image includes generating an output image by convolving and/or cross-correlation the kernel with an input image, the input image being based on the digital image.
66. The method of clause 64 or 65, further including: training the CNN based on training data.
67. The method of clause 66, wherein the training data includes input images and ground truth outputs, and wherein training the CNN based on the training data includes optimizing parameters of one or more kernels of the CNN to minimize a loss between the ground truth outputs and generated outputs of the CNN in response to inputting the input images into the CNN.
68. The method of any of clauses 7 to 67, wherein classifying, using the classifier, the condition of the subject based on the input features includes: generating a classification of the condition by inputting, into a classifier, the input features.
69. The method of clause 68, wherein the classifier includes an ML-based classifier.
70. The method of clause 69, further including: training the ML-based classifier using training data, the training data including population features of population samples obtained from a population omitting the subject.
71. The method of clause 70, further including: identifying sequence read data of the population samples; generating the population features by transforming the sequence read data of the population samples into the alternative domain.
72. The method of clause 70 or 71, wherein training the ML-based classifier using training data includes performing supervised learning on the ML-based classifier.
73. The method of any of clauses 70 to 72, wherein training the ML-based classifier using the training data includes performing unsupervised learning on the ML-based classifier.
74. The method of any of clauses 70 to 73, wherein training the ML-based classifier using the training data includes optimizing parameters of the ML-based classifier using the training data.
75. The method of any of clauses 69 to 74, wherein the ML-based classifier includes at least one of a: an artificial neural network (ANN); a logistic regression model; a random forest model, a decision tree; a k-nearest neighbor (KNN) model; a support vector machine (SVM); or a naïve Bayes classifier.
76. The method of any of clauses 7 to 75, wherein classifying, using the classifier, the condition of the subject based on the input features includes: predicting whether the subject has a pathological condition.
77. The method of clause 76, wherein the pathological condition includes at least one of a cancer, a genetic disorder, diabetes, hypertension, heart disease, a respiratory disease, an infectious disease, or an autoimmune disease.
78. The method of any of clauses 7 to 77, wherein the condition includes at least one of: a predicted pathologic condition of the subject; a predicted pathologic condition subtype of the subject; a metastasis profile of the subject; a predicted survivability of the subject; a predicted symptom of the subject; a predicted effective therapy to treat the predicted pathologic condition of the subject; a predicted resistance of the subject to a treatment of the predicted pathologic condition; a general health of the subject; a genomic age of the subject; a risk of the subject developing the predicted pathologic condition; a predicted stage of the predicted pathologic condition of the subject; a predicted grade of the predicted pathologic condition of the subject; or a predicted Eastern Cooperative Oncology Group (ECOG) performance status of the subject.
79. The method of any of clauses 7 to 78, wherein the condition includes a health metric and/or a disease metric of the subject.
80. The method of any of clauses 7 to 79, wherein the condition includes a likelihood that the subject will develop a disease.
81. The method of any of clauses 7 to 80, further including: applying a filter to the transformed data.
82. The method of clause 81, wherein the filter includes a high pass filter and/or a low pass filter.
83. The method of clause 81 or 82, wherein the filter includes at least one of a Butterworth filter, a Chebyshev filter, a finite impulse response (FIR) filter, or an infinite impulse response (IIR) filter.
84. The method of any of clauses 81 to 83, wherein applying the filter to the transformed data includes multiplying the filter with the transformed data.
85. The method of any of clauses 7 to 84, further including: generating, based on the condition, a genomic profile of the subject.
86. The method of clause 85, wherein the genomic profile includes results from at least one of: a comprehensive genomic profiling test; a whole genome sequencing (WGS) test; a whole exome sequencing (WES) test; a gene expression profiling test; a cancer hotspot panel test; a DNA methylation test; a DNA fragmentation test; or an RNA fragmentation test.
87. The method of clause 85 or 86, wherein the genomic profile of the subject includes: results from a nucleic acid sequencing-based test.
88. The method of any of clauses 85 to 87, further including: selecting, based on the genomic profile and/or the condition, an anticancer agent for administration to the subject.
89. The method of clause 88, further including: administering the anticancer agent to the subject.
90. The method of any of clauses 85 to 89, further including: applying, based on the genomic profile, an anticancer therapy to the subject.
91. The method of clause 90, wherein the anticancer therapy includes at least one of chemotherapy, radiation therapy, immunotherapy, a targeted therapy, or surgery.
92. The method of any of clauses 7 to 91, further including: identifying, based on the condition, a suggested treatment decision for the subject.
93. The method of clause 92, wherein the suggested treatment decision includes radiotherapy and/or chemotherapy.
94. The method of any of clauses 7 to 93, further including: generating a report indicating the condition; and outputting the report.
95. The method of clause 94, wherein outputting the report includes: transmitting data indicating the report to an external device.
96. The method of clause 95, wherein the external device is associated with the subject and/or a healthcare provider.
97. The method of clause 95 or 96, wherein the data is transmitted over one or more communication networks.
98. The method of any of clauses 95 to 97, wherein the data is transmitted over a peer-to-peer connection.
99. The method of any of clauses 94 to 98, wherein outputting the report includes: visually presenting, by a display, the report.
100. The method of any of clauses 94 to 99, further including: determining, based on the condition, one or more therapies to treat the condition of the subject, wherein the report further indicates the one or more therapies.
101. The method of clause 100, wherein the condition includes at least one type or subtype of cancer.
102. The method of any of clauses 7 to 101, further including: generating, based on the condition, a therapy for the subject.
103. The method of clause 102, wherein the therapy includes a dosage of one or more therapeutic agents predicted to treat the condition of the subject.
104. The method of any of clauses 7 to 103, further including: determining, based on the condition, whether the subject is eligible for a clinical trial.
105. A system including at least one processor configured to perform the method of any of clauses 7 to 104.
106. A non-transitory computer-readable medium storing instructions for performing the method of any of clauses 7 to 104.
107. A system, including: at least one processor; and memory storing instructions that, when executed by the at least one processor, cause the at least one processor to perform operations including: identifying sequence read data of a sample obtained from a subject, the sequence read data being in a spatial domain corresponding to genomic position; generating transformed data by transforming the sequence read data into an alternative domain; generating input features based on the transformed data; and classifying, using a classifier, a condition of the subject based on the input features.
108. The system of clause 107, further including: a sequencer configured to generate the sequence read data by sequencing a plurality of nucleic acid molecules in the sample.
109. The system of clause 107 or 108, further including: a transceiver configured to transmit data indicating the condition of the subject.
110. The system of any of clauses 107 to 109, further including: an output device configured to output an indication of the condition of the subject.
111. A non-transitory computer readable medium storing instructions for performing operations including: identifying sequence read data of a sample obtained from a subject, the sequence read data being in a spatial domain corresponding to genomic position; generating transformed data by transforming the sequence read data into an alternative domain; generating input features based on the transformed data; and classifying, using a classifier, a condition of the subject based on the input features.

### CONCLUSION

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on.” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy.” Appl Immunohistochem Mol Morphol. 26 (2): e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV). Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation. EGFR exon 20 activation mutation. EGFR exon 20 insertion. EGFR G719 mutation, EGFR L858R mutation. EGFR L861 mutation, EGFR S768 mutation, EGFR T790M mutation, C797 mutation, KIT activating mutation. KRAS activating mutation. MET activating mutation. NRAS activating mutation, PMS2 promoter mutations, among many others, Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data, the sequence read data being in a spatial domain corresponding to genomic position within at least one locus of a genome of the sample;
receiving, at one or more processors, the sequence read data for the plurality of sequence reads;
generating, by the one or more processors, transformed data by performing a Fast Fourier Transform (FFT) on the sequence read data, the transformed data representing the sequence read data in a frequency domain;
generating, by the one or more processors, input features based on the transformed data; and
determining, using a classifier executed by the one or more processors, a condition of the subject based on the input features.

2. The method of claim 1, wherein the plurality of nucleic acid molecules comprise DNA fragments obtained from a liquid biopsy sample obtained from the subject, and
wherein the sequence read data comprises endpoint counts of the DNA fragments at multiple genomic positions within the at least one locus of the genome of the sample.

3. The method of claim 1, wherein generating, by the one or more processors, the input features based on the transformed data comprises at least one of:
inputting, into a convolutional neural network (CNN) configured to detect the input features, an image representing the transformed data;
identifying one or more peaks of the transformed data;
identifying one or more troughs of the transformed data;
determining a first distance metric by comparing the transformed data and first pre-classified data, the first pre-classified data being in the frequency domain and being transformed from sequence read data of a first individual having the condition; or
determining a second distance metric by comparing the transformed data and second pre-classified data, the second pre-classified data being in the frequency domain and transformed from sequence read data of a second individual that does not have the condition.

4. The method of claim 3, wherein determining the first distance metric comprises:
generating first alignment data by cross-correlating and/or convolving the transformed data and the first pre-classified data; and
determining at least one of a peak of the first alignment data or a mean of the first alignment data, and
wherein determining the second distance metric comprises:
generating second alignment data by cross-correlating and/or convolving the transformed data and the second pre-classified data; and
determining at least one of a peak of the second alignment data or a mean of the second alignment data.

5. The method of claim 1, wherein the condition comprises a cancer type or subtype of the subject.

6. The method of claim 1, further comprising at least one of:
removing a high-frequency artifact from the transformed data by multiplying a low-pass filter with the transformed data, or
removing a low-frequency artifact from the transformed data by multiplying a high-pass filter with the transformed data.

7. A method, comprising:
identifying sequence read data of a sample obtained from a subject, the sequence read data being in a spatial domain corresponding to genomic position;
generating transformed data by transforming the sequence read data into an alternative domain;
generating input features based on the transformed data; and
classifying, using a classifier, a condition of the subject based on the input features.

8. The method of claim 7, wherein the sequence read data comprises at least one of:
endpoint counts of fragments in the sample at multiple genomic positions;
read depth of the fragments in the sample at the multiple genomic positions;
lengths of the fragments in the sample;
a quantity of variants present in the fragments in the sample at the multiple genomic positions; or
a presence of the variants in the fragments in the sample at the multiple genomic positions.

9. The method of claim 7, wherein generating the input features further comprises:
identifying, by analyzing the sequence read data, one or more pathogenic variants in at least one of POLE, TP53, CTNNNB1, L1CAM, PTEN, ERBB2, PMS2, MSH2, MSH6, MLH1, an estrogen receptor (ER) gene, or a progesterone receptor (PR) gene.

10. The method of claim 7, wherein generating the transformed data by transforming the sequence read data into the alternative domain comprises:
performing at least one of a Fourier transform, a short-time Fourier transform (STFT), a discrete Fourier transform (DFT), a Hartley transform, Laplace transform, a Mellin transform, or a Wavelet transform on the sequence read data.

11. The method of claim 7, wherein generating the input features based on the transformed data comprises:
generating alignment data by cross-correlating and/or convolving the transformed data and pre-classified data, the pre-classified data being in the alternate domain; and
determining a distance metric based on the alignment data.

12. The method of claim 11, wherein the pre-classified data is based on a sample obtained from an individual that has the condition, has a predetermined subtype of the condition, or that lacks the condition.

13. The method of claim 7, wherein generating the input features based on the transformed data comprises:
generating a digital image based on the transformed data, and
extracting the input features from the digital image by inputting the digital image into a convolutional neural network (CNN).

14. The method of claim 13, wherein the CNN comprises multiple layers, an individual layer among the multiple layers comprising a kernel defined by one or more parameters, and
wherein extracting the input features from the digital image comprises generating an output image by convolving and/or cross-correlation the kernel with an input image, the input image being based on the digital image.

15. The method of claim 7, wherein classifying, using the classifier, the condition of the subject based on the input features comprises:
generating a classification of the condition by inputting, into a classifier, the input features.

16. The method of claim 15, wherein the classifier comprises at least one of a:
an artificial neural network (ANN);
a logistic regression model;
a random forest model,
a decision tree;
a k-nearest neighbor (KNN) model;
a support vector machine (SVM); or
a naïve Bayes classifier.

17. The method of claim 7, wherein classifying, using the classifier, the condition of the subject based on the input features comprises:
predicting whether the subject has a pathological condition comprising at least one of a cancer, a genetic disorder, diabetes, hypertension, heart disease, a respiratory disease, an infectious disease, or an autoimmune disease.

18. The method of claim 7, wherein the condition comprises at least one of a health metric of the subject, a disease metric of the subject, or a likelihood that the subject will develop a disease.

19. The method of claim 7, further comprising:
applying a filter to the transformed data, the filter comprising a high pass filter and/or a low pass filter.

20. A system, comprising:
a sequencer configured to generate sequence read data by sequencing a plurality of nucleic acid molecules in a sample;
at least one processor; and
memory storing instructions that, when executed by the at least one processor, cause the at least one processor to perform operations comprising:
identifying the sequence read data of the sample obtained from a subject, the sequence read data being in a spatial domain corresponding to genomic position;
generating transformed data by transforming the sequence read data into an alternative domain;
generating input features based on the transformed data; and
classifying, using a classifier, a condition of the subject based on the input features.
